SEQUENCE LISTING (54 sequences)

SEQ ID NO: 1 - protein, 1612 aa, Homo sapiens
MSAGGRDEERRKLADIIHHWNANRLDLFEISQPTEDLEFHGVMRFYFQDKAAGNFATKCIRVSSTATTQDVIETLAEKFRPDMRMLSSPKYSLYEVHVSGERRLDIDEKPLVVQLNWNKDDREGRFVLKNENDAIPPKAQSNGPEKQEKEGVIQNFKRTLSKKEKKEKKKREKEALRQASDKDDRPFQGEDVENSRLAAEVYKDMPETSFTRTISNPEVVMKRRRQQKLEKRMQEFRSSDGRPDSGGTLRIYADSLKPNIPYKTILLSTTDPADFAVAEALEKYGLEKENPKDYCIARVMLPPGAQHSDEKGAKEIILDDDECPLQIFREWPSDKGILVFQLKRRPPDHIPKKTKKHLEGKTPKGKERADGSVYGSTLPPEKLPYLVELSPDGSDSRDKPKLYRLQLSVTEVGTEKLDDNSIQLFGPGIQPHHCDLTNMDGVVTVTPRSMDAETYVEGQRISETTMLQSGMKVQFGASHVFKFVDPSQDHALAKRSVDGGLMVKGPRHKPGIVQETTFDLGGDIHSGTALPTSKSTTRLDSDRVSSASSTAERGMVKPMIRVEQQPDYRRQESRTQDASGPELILPASIEFRESSEDSFLSAIINYTNSSTVHFKLSPTYVLYMACRYVLSNQYRPDISPTERTHKVIAVVNKMVSMMEGVIQKQKNIAGALAFWMANASELLNFIKQDRDLSRITLDAQDVLAHLVQMAFKYLVHCLQSELNNYMPAFLDDPEENSLQRPKIDDVLHTLTGAMSLLRRCRVNAALTIQLFSQLFHFINMWLFNRLVTDPDSGLCSHYWGAIIRQQLGHIEAWAEKQGLELAADCHLSRIVQATTLLTMDKYAPDDIPNINSTCFKLNSLQLQALLQNYHCAPDEPFIPTDLIENVVTVAENTADELARSDGREVQLEEDPDLQLPFLLPEDGYSCDVVRNIPNGLQEFLDPLCQRGFCRLIPHTRSPGTWTIYFEGADYESHLLRENTELAQPLRKEPEIITVTLKKQNGMGLSIVAAKGAGQDKLGIYVKSVVKGGAADVDGRLAAGDQLLSVDGRSLVGLSQERAAELMTRTSSVVTLEVAKQGAIYHGLATLLNQPSPMMQRISDRRGSGKPRPKSEGFELYNNSTQNGSPESPQLPWAEYSEPKKLPGDDRLMKNRADHRSSPNVANQPPSPGGKSAYASGTTAKITSVSTGNLCTEEQTPPPRPEAYPIPTQTYTREYFTFPASKSQDRMAPPQNQWPNYEEKPHMHTDSNHSSIAIQRVTRSQEELREDKAYQLERHRIEAAMDRKSDSDMWINQSSSLDSSTSSQEHLNHSSKSVTPASTLTKSGPGRWKTPAAIPATPVAVSQPIRTDLPPPPPPPPVHYAGDFDGMSMDLPLPPPPSANQIGLPSAQVAAAERRKREEHQRWYEKEKAPLEEERERKRREQERKLGQMRTQSLNPAPFSPLTAQQMKPEKPSTLQRPQETVIRELQPQQQPRTIERRDLQYITVSKEELSSGDSLSPDPWKRDAKEKLEKQQQMHIVDMLSKEIQELQSKPDRSAEESDRLRKLMLEWQFQKRLQESKQKDEDDEEEEDDDVDTMLIMQRLEAERRARVKGGVLWLCPSVVPILASACFPWG

SEQ ID NO: 2 - protein, 403 aa, Homo sapiens
MDRSKENCISGPVKATAPVGGPKRVLVTQQIPCQNPLPVNSGQAQRVLCPSNSSQRVPLQAQKLVSSHKPVQNQKQKQLQATSVPHPVSRPLNNTQKSKQPLPSAPENNPEEELASKQKNEESKKRQWALEDFEIGRPLGKGKFGNVYLAREKQSKFILALKVLFKAQLEKAGVEHQLRREVEIQSHLRHPNILRLYGYFHDATRVYLILEYAPLGTVYRELQKLSKFDEQRTATYITELANALSYCHSKRVIHRDIKPENLLLGSAGELKIADFGWSVHAPSSRRTTLCGTLDYLPPEMIEGRMHDEKVDLWSLGVLCYEFLVGKPPFEANTYQETYKRISRVEFTFPDFVTEGARDLISRLLKHNPSQRPMLREVLEHPWITANSSKPSNCQNKESASKQS

SEQ ID NO: 3 - protein, 907 aa, Homo sapiens
MTAVHAGNINFKWDPKSLEIRTLAVERLLEPLVTQVTTLVNTNSKGPSNKKRGRSKKAHVLAASVEQATENFLEKGDKIAKESQFLKEELVVAVEDVRKQGDLMKAAAGEFADDPCSSVKRGNMVRAARALLSAVTRLLILADMADVYKLLVQLKVVEDGILKLRNAGNEQDLGNQYKALKPEVDKLNIMAAKRQQELKDVGHRDQMAAARGILQSNVPILYTASQACLQHPDVAAYKANRDLIYKQLQQAVTGISNAAQATASDDASQHQGGGGGELAYALNNFDKQIIVDPLSFSEERFRPSLEERLESIISGAALMADSSCTRDDRRERIVAECNAVRQACRTCVSEYMGNAGRKERSDALNSAIDKMTKKTRDLRRQLRKAVMDHVSDSFLETNVPLLVLIEAAKNGNEKEVKEYAQVFREHANKLIEVANLACSISNNEEGVKLVRMSASQLEAGCPQVINAATWALAPKPQSKLAQENMDLFKEQWEKQVRVLTDAVDDITSIDDFLAVSENHILEDVNKCVIALQEKDVDGLDRTAGAIRGRAARVIHVVTSEMDNYEPGVYTEKVLEATKLLSNTVMPRFTEQVEAAVEALSSDPAQPMDENEFIDASRLVYDGIRDIRKAVLMIRTPEELDDSDFETEDFDVRSETSVQTEDDQLIAGQSARAIMAQLPQEQKAKIREQVASFQEEKSKLDAEVSKWDDSGNDIIVLAKQMCMIMMEMTDFTRGKGPLKNTSDVISAAKKIAEAGSRMDKLGRTIRDHCPDSACKQDLLAYLQRIALYCHQLNICSKVKAEVQNLGGELVVSGVDSAMSLIQAAKNLMNAVVQTVKASYVASTKYQKSQGMASLNLPAVSMKMKAPEKKPLVKREKQDETQTKIKRASQKKHVNPVQALSEFKAMDSI

SEQ ID NO: 4 - protein, 781 aa, Homo sapiens
MATQADLMELDMAMEPDRKAAVSHWQQQSYLDSGIHSGATTTAPSLSGKGNPEEEDVDTSQVLYEWEQGFSQSFTQEQVADIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEPLGYRQDDPSYRSFHSGGYGQDALGMDPMMEHEMGGHHPGADYPVDGLPDLGHAQDLMDGLPPGDSNQLAWFDTDL

SEQ ID NO: 5 - protein, 239 aa, Homo sapiens
MAHAGRTGYDNREIVMKYIHYKLSQRGYEWDAGDVGAAPPGAAPAPGIFSSQPGHTPHPAASRDPVARTSPLQTPAAPGAAAGPALSPVPPVVHLTLRQAGDDFSRRYRRDFAEMSSQLHLTPFTARGRFATVVEELFRDGVNWGRIVAFFEFGGVMCVESVNREMSPLVDNIALWMTEYLNRHLHTWIQDNGGWDAFVELYGPSMRPLFDFSWLSLKTLLSLALVGACITLGAYLGHK

SEQ ID NO: 6 - protein, 295 aa, Homo sapiens
MEHQLLCCEVETIRRAYPDANLLNDRVLRAMLKAEETCAPSVSYFKCVQKEVLPSMRKIVATWMLEVCEEQKCEEEVFPLAMNYLDRFLSLEPVKKSRLQLLGATCMFVASKMKETIPLTAEKLCIYTDNSIRPEELLQMELLLVNKLKWNLAAMTPHDFIEHFLSKMPEAEENKQIIRKHAQTFVALCATDVKFISNPPSMVAAGSVVAAVQGLNLRSPNNFLSYYRLTRFLSRVIKCDPDCLRACQEQIEALLESSLRQAQQNMDPKAAEEEEEEEEEVDLACTPTDVRDVDI

SEQ ID NO: 7 - protein, 395 aa, Homo sapiens
MKEDGGAEFSARSRKRKANVTVFLQDPDEEMAKIDRTARDQCGSQPWDNNAVCADPCSLIPTPDKEDDDRVYPNSTCKPRIIAPSRGSPLPVLSWANREEVWKIMLNKEKTYLRDQHFLEQHPLLQPKMRAILLDWLMEVCEVYKLHRETFYLAQDFFDRYMATQENVVKTLLQLIGISSLFIAAKLEEIYPPKLHQFAYVTDGACSGDEILTMELMIMKALKWRLSPLTIVSWLNVYMQVAYLNDLHEVLLPQYPQQIFIQIAELLDLCVLDVDCLEFPYGILAASALYHFSSSELMQKVSGYQWCDIENCVKWMVPFAMVIRETGSSKLKHFRGVADEDAHNIQTHRDSLDLLDKARAKKAMLSEQNRASPLPSGLLTPPQSGKKQSSGPEMA

SEQ ID NO: 8 - protein, 590 aa, Homo sapiens
MSRQSSVSFRSGGSRSFSTASAITPSVSRTSFTSVSRSGGGGGGGFGRVSLAGACGVGGYGSRSLYNLGGSKRISISTRGGSFRNRFGAGAGGGYGFGGGAGSGFGFGGGAGGGFGLGGGAGFGGGFGGPGFPVCPPGGIQEVTVNQSLLTPLNLQIDPSIQRVRTEEREQIKTLNNKFASFIDKVRFLEQQNKVLDTKWTLLQEQGTKTVRQNLEPLFEQYINNLRRQLDSIVGERGRLDSELRNMQDLVEDFKNKYEDEINKRTTAENEFVMLKKDVDAAYMNKVELEAKVDALMDEINFMKMFFDAELSQMQTHVSDTSVVLSMDNNRNLDLDSIIAEVKAQYEEIANRSRTEAESWYQTKYEELQQTAGRHGDDLRNTKHEITEMNRMIQRLRAEIDNVKKQCANLQNAIADAEQRGELALKDARNKLAELEEALQKAKQDMARLLREYQELMNTKLALDVEIATYRKLLEGEECRLSGEGVGPVNISVVTSSVSSGYGSGSGYGGGLGGGLGGGLGGGLAGGSSGSYYSSSSGGVGLGGGLSVGGSGFSASSGRGLGVGFGSGGGSSSSVKFVSTTSSSRKSFKS

SEQ ID NO: 9 - protein, 483 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 10 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 11 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA

SEQ ID NO: 12 - protein, 1255 aa, Homo sapiens [sequence omitted]

SEQ ID NO: 13 - protein, 1342 aa, Homo sapiens
MRANDALQVLGLLFSLARGSEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMGGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGGSCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 14 - protein, 1308 aa, Homo sapiens
MKPATGLWVWVSLLVAAGTVQPSDSQSVCAGTENKLSSLSDLEQQYRALRKYYENCEVVMGNLEITSIEHNRDLSFLRSVREVTGYVLVALNQFRYLPLENLRIIRGTKLYEDRYALAIFLNYRKDGNFGLQELGLKNLTEILNGGVYVDQNKFLCYADTIHWQDIVRNPWPSNLTLVSTNGSSGCGRCHKSCTGRCWGPTENHCQTLTRTVCAEQCDGRCYGPYVSDCCHRECAGGCSGPKDTDCFACMNFNDSGACVTQCPQTFVYNPTTFQLEHNFNAKYTYGAFCVKKCPHNFVVDSSSCVRACPSSKMEVEENGIKMCKPCTDICPKACDGIGTGSLMSAQTVDSSNIDKFINCTKINGNLIFLVTGIHGDPYNAIEAIDPEKLNVFRTVREITGFLNIQSWPPNMTDFSVFSNLVTIGGRVLYSGLSLLILKQQGITSLQFQSLKEISAGNIYITDNSNLCYYHTINWTTLFSTINQRIVIRDNRKAENCTAEGMVCNHLCSSDGCWGPGPDQCLSCRRFSRGRICIESCNLYDGEFREFENGSICVECDPQCEKMEDGLLTCHGPGPDNCTKCSHFKDGPNCVEKCPDGLQGANSFIFKYADPDRECHPCHPNCTQGCNGPTSHDCIYYPWTGHSTLPQHARTPLIAAGVIGGLFILVIVGLTFAVYVRRKSIKKKRALRRFLETELVEPLTPSGTAPNQAQLRILKETELKRVKVLGSGAFGTVYKGIWVPEGETVKIPVAIKILNETTGPKANVEFMDEALIMASMDHPHLVRLLGVCLSPTIQLVTQLMPHGCLLEYVHEHKDNIGSQLLLNWCVQIAKGMMYLEERRLVHRDLAARNVLVKSPNHVKITDFGLARLLEGDEKEYNADGGKMPIKWMALECIHYRKFTHQSDVWSYGVTIWELMTFGGKPYDGIPTREIPDLLEKGERLPQPPICTIDVYMVMVKCWMIDADSRPKFKELAAEFSRMARDPQRYLVIQGDDRMKLPSPNDSKFFQNLLDEEDLEDMMDAEEYLVPQAFNIPPPIYTSRARIDSNRSEIGHSPPPAYTPMSGNQFVYRDGGFAAEQGVSVPYRAPTSTIPEAPVAQGATAEIFDDSCCNGTLRKPVAPHVQEDSSTQRYSADPTVFAPERSPRGELDEEGYMTPMRDKPKQEYLNPVEENPFVSRRKNGDLQALDNPEYHNASNGPPKAEDEYVNEPLYLNTFANTLGKAEYLKNNILSMPEKAKKAFDNPDYWNHSLPPRSTLQHPDYLQEYSTKYFYKQNGRIRPIVAENPEYLSEFSLKPGTVLPPPPYRHRNTVV

SEQ ID NO: 15 - protein, 595 aa, Homo sapiens
MTMTLHTKASGMALLHQIQGNELEPLNRPQLKIPLERPLGEVYLDSSKPAVYNYPEGAAYEFNAAAAANAQVYGQTGLPYGPGSEAAAFGSNGLGGFPPLNSVSPSPLMLLHPPPQLSPFLQPHGQQVPYYLENEPSGYTVREAGPPAFYRPNSDNRRQGGRERLASTNDKGSMAMESAKETRYCAVCNDYASGYHYGVWSCEGCKAFFKRSIQGHNDYMCPATNQCTIDKNRRKSCQACRLRKCYEVGMMKGGIRKDRRGGRMLKHKRQRDDGEGRGEVGSAGDMRAANLWPSPLMIKRSKKNSLALSLTADQMVSALLDAEPPILYSEYDPTRPFSEASMMGLLTNLADRELVHMINWAKRVPGFVDLTLHDQVHLLECAWLEILMIGLVWRSMEHPVKLLFAPNLLLDRNQGKCVEGMVEIFDMLLATSSRFRMMNLQGEEFVCLKSIILLNSGVYTFLSSTLKSLEEKDHIHRVLDKITDTLIHLMAKAGLTLQQQHQRLAQLLLILSHIRHMSNKGMEHLYSMKCKNVVPLYDLLLEMLDAHRLHAPTSRGGASVEETDQSHLATAGSTSSHSLQKYYITGEAEGFPATV

SEQ ID NO: 16 - protein, 820 aa, Homo sapiens
MWSWKCLLFWAVLVTATLCTARPSPTLPEQAQPWGAPVEVESFLVHPGDLLQLRCRLRDDVQSINWLRDGVQLAESNRTRITGEEVEVQDSVPADSGLYACVTSSPSGSDTTYFSVNVSDALPSSEDDDDDDDSSSEEKETDNTKPNPVAPYWTSPEKMEKKLHAVPAAKTVKFKCPSSGTPNPTLRWLKNGKEFKPDHRIGGYKVRYATWSIIMDSVVPSDKGNYTCIVENEYGSINHTYQLDVVERSPHRPILQAGLPANKTVALGSNVEFMCKVYSDPQPHIQWLKHIEVNGSKIGPDNLPYVQILKTAGVNTTDKEMEVLHLRNVSFEDAGEYTCLAGNSIGLSHHSAWLTVLEALEERPAVMTSPLYLEIIIYCTGAFLISCMVGSVIVYKMKSGTKKSDFHSQMAVHKLAKSIPLRRQVTVSADSSASMNSGVLLVRPSRLSSSGTPMLAGVSEYELPEDPRWELPRDRLVLGKPLGEGCFGQVVLAEAIGLDKDKPNRVTKVAVKMLKSDATEKDLSDLISEMEMMKMIGKHKNIINLLGACTQDGPLYVIVEYASKGNLREYLQARRPPGLEYCYNPSHNPEEQLSSKDLVSCAYQVARGMEYLASKKCIHRDLAARNVLVTEDNVMKIADFGLARDIHHIDYYKKTTNGRLPVKWMAPEALFDRIYTHQSDVWSFGVLLWEIFTLGGSPYPGVPVEELFKLLKEGHRMDKPSNCTNELYMMMRDCWHAVPSQRPTFKQLVEDLDRIVALTSNQEYLDLSMPLDQYSPSFPDTRSSTCSSGEDSVFSHEPLPEEPCLPRHPAQLANGGLKRR

SEQ ID NO: 17 - protein, 147 aa, Homo sapiens
MSFRFGQHLIKPSVVFLKTELSFALVNRKPVVPGHVLVCPLRPVERFHDLRPDEVADLFQTTQRVGTVVEKHFHGTSLTFSMQDGPEAGQTVKHVHVHVLPRKAGDFHRNDSIYEELQKHDKEDFPASWRSEEEMAAEAAALRVYFQ

SEQ ID NO: 18 - protein, 444 aa, Homo sapiens
MEVTADQPRWVSHHHPAVLNGQHPDTHHPGLSHSYMDAAQYPLPEEVDVLFNIDGQGNHVPPYYGNSVRATVQRYPPTHHGSQVCRPPLLHGSLPWLDGGKALGSHHTASPWNLSPFSKTSIHHGSPGPLSVYPPASSSSLSGGHASPHLFTFPPTPPKDVSPDPSLSTPGSAGSARQDEKECLKYQVPLPDSMKLESSHSRGSMTALGGASSSTHHPITTYPPYVPEYSSGLFPPSSLLGGSPTGFGCKSRPKARSSTEGRECVNCGATSTPLWRRDGTGHYLCNACGLYHKMNGQNRPLIKPKRRLSAARRAGTSCANCQTTTTTLWRRNANGDPVCNACGLYYKLHNINRPLTMKKEGIQTRNRKMSSKSKKCKKVHDSLEDFPKNSSFNPAALSRHMSSLSHISPFSHSSHMLTTPTPMHPPSSLSFGPHHPSSMVTAMG

SEQ ID NO: 19 - protein, 3256 aa, Homo sapiens
MWPTRRLVTIKRSGVDGPHFPLSLSTCLFGRGIECDIRIQLPVVSKQHCKIEIHEQEAILHNFSSTNPTQVNGSVIDEPVRLKHGDVITIIDRSFRYENESLQNGRKSTEFPRKIREQEPARRVSRSSFSSDPDEKAQDSKAYSKITEGKVSGNPQVHIKNVKEDSTADDSKDSVAQGTTNVHSSEHAGRNGRNAADPISGDFKEISSVKLVSRYGELKSVPTTQCLDNSKKNESPFWKLYESVKKELDVKSQKENVLQYCRKSGLQTDYATEKESADGLQGETQLLVSRKSRPKSGGSGHAVAEPASPEQELDQNKGKGRDVESVQTPSKAVGASFPLYEPAKMKTPVQYSQQQNSPQKHKNKDLYTTGRRESVNLGKSEGFKAGDKTLTPRKLSTRNRTPAKVEDAADSATKPENLSSKTRGSIPTDVEVLPTETEIHNEPFLTLWLTQVERKIQKDSLSKPEKLGTTAGQMCSGLPGLSSVDINNFGDSINESEGIPLKRRRVSFGGHLRPELFDENLPPNTPLKRGEAPTKRKSLVMHTPPVLKKIIKEQPQPSGKQESGSEIHVEVKAQSLVISPPAPSPRKTPVASDQRRRSCKTAPASSSKSQTEVPKRGGERVATCLQKRVSISRSQHDILQMICSKRRSGASEANLIVAKSWADVVKLGAKQTQTKVIKHGPQRSMNKRQRRPATPKKPVGEVHSQFSTGHANSPCTIIIGKAHTEKVHVPARPYRVLNNFISNQKMDFKEDLSGIAEMFKTPVKEQPQLTSTCHIAISNSENLLGKQFQGTDSGEEPLLPTSESFGGNVFFSAQNAAKQPSDKCSASPPLRRQCIRENGNVAKTPRNTYKMTSLETKTSDTETEPSKTVSTVNRSGRSTEFRNIQKLPVESKSEETNTEIVECILKRGQKATLLQQRREGEMKEIERPFETYKENIELKENDEKMKAMKRSRTWGQKCAPMSDLTDLKSLPDTELMKDTARGQNLLQTQDHAKAPKSEKGKITKMPCQSLQPEPINTPTHTKQQLKASLGKVGVKEELLAVGKFTRTSGETTHTHREPAGDGKSIRTFKESPKQILDPAARVTGMKKWPRTPKEEAQSLEDLAGFKELFQTPGPSEESMTDEKTTKIACKSPPPESVDTPTSTKQWPKRSLRKADVEEEFLALRKLTPSAGKAMLTPKPAGGDEKDIKAFMGTPVQKLDLAGTLPGSKRQLQTPKEKAQALEDLAGFKELFQTPGHTEELVAAGKTTKIPCDSPQSDPVDTPTSTKQRPKRSIRKADVEGELLACRNLMPSAGKAMHTPKPSVGEEKDIIIFVGTPVQKLDLTENLTGSKRRPQTPKEEAQALEDLTGFKELFQTPGHTEEAVAAGKTTKMPCESSPPESADTPTSTRRQPKTPLEKRDVQKELSALKKLTQTSGETTHTDKVPGGEDKSINAFRETAKQKLDPAASVTGSKRHPKTKEKAQPLEDLAGWKELFQTPVCTDKPTTHEKTTKIACRSQPDPVDTPTSSKPQSKRSLRKVDVEEEFFALRKRTPSAGKAMHTPKPAVSGEKNIYAFMGTPVQKLDLTENLTGSKRRLQTPKEKAQALEDLAGFKELFQTRGHTEESMTNDKTAKVACKSSQPDLDKNPASSKRRLKTSLGKVGVKEELLAVGKLTQTSGETTHTHTEPTGDGKSMKAFMESPKQILDSAASLTGSKRQLRTPKGKSEVPEDLAGFIELFQTPSHTKESMTNEKTTKVSYRASQPDLVDTPTSSKPQPKRSLRKADTEEEFLAFRKQTPSAGKAMHTPKPAVGEEKDINTFLGTPVQKLDQPGNLPGSNRRLQTRKEKAQALEELTGFRELFQTPCTDNPTADEKTTKKILCKSPQSDPADTPTNTKQRPKRSLKKADVEEEFLAFRKLTPSAGKAMHTPKAAVGEEKDINTFVGTPVEKLDLLGNLPGSKRRPQTPKEKAKALEDLAGFKELFQTPGHTEESMTDDKITEVSCKSPQPDPVKTPTSSKQRLKISLGKVGVKEEVLPVGKLTQTSGKTTQTHRETAGDGKSIKAFKESAKQMLDPANYGTGMERWPRTPKEEAQSLEDLAGFKELFQTPDHTEESTTDDKTTKIACKSPPPESMDTPTSTRRRPKTPLGKRDIVEELSALKQLTQTTHTDKVPGDEDKGINVFRETAKQKLDPAASVTGSKRQPRTPKGKAQPLEDLAGLKELFQTPVCTDKPTTHEKTTKIACRSPQPDPVGTPTIFKPQSKRSLRKADVEEESLALRKRTPSVGKAMDTPKPAGGDEKDMKAFMGTPVQKLDLPGNLPGSKRWPQTPKEKAQALEDLAGFKELFQTPGTDKPTTDEKTTKIACKSPQPDPVDTPASTKQRPKRNLRKADVEEEFLALRKRTPSAGKAMDTPKPAVSDEKNINTFVETPVQKLDLLGNLPGSKRQPQTPKEKAEALEDLVGFKELFQTPGHTEESMTDDKITEVSCKSPQPESFKTSRSSKQRLKIPLVKVDMKEEPLAVSKLTRTSGETTQTHTEPTGDSKSIKAFKESPKQILDPAASVTGSRRQLRTRKEKARALEDLVDFKELFSAPGHTEESMTIDKNTKIPCKSPPPELTDTATSTKRCPKTRPRKEVKEELSAVERLTQTSGQSTHTHKEPASGDEGIKVLKQRAKKKPNPVEEEPSRRRPRAPKEKAQPLEDLAGFTELSETSGHTQESLTAGKATKIPCESPPLEVVDTTASTKRHLRTRVQKVQVKEEPSAVKFTQTSGETTDADKEPAGEDKGIKALKESAKQTPAPAASVTGSRRRPRAPRESAQAIEDLAGFKDPAAGHTEESMTDDKTTKIPCKSSPELEDTATSSKRRPRTRAQKVEVKEELLAVGKLTQTSGETTHTDKEPVGEGKGTKAFKQPAKRNVDAEDVIGSRRQPRAPKEKAQPLEDLASFQELSQTPGHTEELANGAADSFTSAPKQTPDSGKPLKISRRVLRAPKVEPVGDVVSTRDPVKSQSKSNTSLPPLPFKRGGGKDGSVTGTKRLRCMPAPEEIVEELPASKKQRVAPRARGKSSEPVVIMKRSLRTSAKRIEPAEELNSNDMKTNKEEHKLQDSVPENKGISLRSRRQDKTEAEQQITEVFVLAERIEINRNEKKPMKTSPEMDIQNPDDGARKPIPRDKVTENKRCLRSARQNESSQPKVAEESGGQKSAKVLMQNQKGKGEAGNSDSMCLRSRKTKSQPAASTLESKSVQRVTRSVKRCAENPKKAEDNVCVKKITTRSHRDSEDI

SEQ ID NO: 20 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 21 - protein, 393 aa, Homo sapiens [sequence omitted]

SEQ ID NO: 22 - protein, 829 aa, Homo sapiens [sequence omitted]

SEQ ID NO: 23 - protein, 933 aa, Homo sapiens
MTELKAKGPRAPHVAGGPPSPEVGSPLLCRPAAGPFPGSQTSDTLPEVSAIPISLDGLLFPRPCQGQDPSDEKTQDQQSLSDVEGAYSRAEATRGAGGSSSSPPEKDSGLLDSVLDTLLAPSGPGQSQPSPPACEVTSSWCLFGPELPEDPPAAPATQRVLSPLMSRSGCKVGDSSGTAAAHKVLPRGLSPARQLLLPASESPHWSGAPVKPSPQAAAVEVEEEDGSESEESAGPLLKGKPRALGGAAAGGGAAAVPPGAAAGGVALVPKEDSRFSAPRVALVEQDAPMAPGRSPLATTVMDFIHVPILPLNHALLAARTRQLLEDESYDGGAGAASAFAPPRSSPCASSTPVAVGDFPDCAYPPDAEPKDDAYPLYSDFQPPALKIKEEEEGAEASARSPRSYLVAGANPAAFPDFPLGPPPPLPPRATPSRPGEAAVTAAPASASVSSASSSGSTLECILYKAEGAPPQQGPFAPPPCKAPGASGCLLPRDGLPSTSASAAAAGAAPALYPALGLNGLPQLGYQAAVLKEGLPQVYPPYLNYLRPDSEASQSPQYSFESLPQKICLICGDEASGCHYGVLTCGSCKVFFKRAMEGQHNYLCAGRNDCIVDKIRRKNCPACRLRKCCQAGMVLGGRKFKKFNKVRVVRALDAVALPQPVGVPNESQALSQRFTFSPGQDIQLIPPLINLLMSIEPDVIYAGHDNTKPDTSSSLLTSLNQLGERQLLSVVKWSKSLPGFRNLHIDDQITLIQYSWMSLMVFGLGWRSYKHVSGQMLYFAPDLILNEQRMKESSFYSLCLTMWQIPQEFVKLQVSQEEFLCMKVLLLLNTIPLEGLRSQTQFEEMRSSYIRELIKAIGLRQKGVVSSSQRFYQLTKLLDNLHDLVKQLHLYCLNTFIQSRALSVEFPEMMSEVIAAQLPKILAGMVKPLLFHKK

SEQ ID NO: 24 - protein, 805 aa, Homo sapiens
MAFSPWQILSPVQWAKWTWSAVRGGAAGEDEAGGPEGDPEEEDSQAETKSLSFSSDSEGNFETPEAETPIRSPFKESCDPSLGLAGPGAKSQESQEADEQLVAEVVEKCSSKTCSKPSENEVPQQAIDSHSVKNFREEPEHDFSKISIVRPFSIETKDSTDISAVLGTKAAHGCVTAVSGKALPSSPPDALQDEAMTEGSMGVTLEASAEADLKAGNSCPELVPSRRSKLRKPKPVPLRKKAIGGEFSDTNAAVEGTPLPKASYHFSPEELDENTSPLLGDARFQKSPPDIKETPGTLSSDTNDSGVELGEESRSSPLKLEFDFTEDTGNIEARKALPRKLGRKLGSTLTPKIQKDGISKSAGLEQPTDPVARDGPLSQTSSKPDPSQWESPSFNPFGSHSVLQNSPPLSSEGSYHFDPDNFDESMDPFKPTTTLTSSDFCSPTGNHVNEILESPKKAKSRLITSGCKVKKHETQSLALDACSRDEGAVISQISDISNRDGHATDEEKLASTSCGQKSAGAEVKGEPEEDLEYFECSNVPVSTINHAFSSSEAGIEKETCQKMEEDGSTVLGLLESSAEKAPVSVSCGGESPLDGICLSESDKTAVLTLIREEIITKEIEANEWKKKYEETRQEVLEMRKIVAEYEKTIAQMIEDEQRTSMTSQKSFQQLTMEKEQALADLNSVERSLSDLFRRYENLKGVLEGFKKNEEALKKCAQDYLARVKQEEQRYQALKIHAEEKLDKANEEIAQVRTKAKAESAALHAGLRKEQMKVESLERALQQKNQEIEELTKICDELIAKLGKTD

SEQ ID NO: 25 - protein, 1026 aa, Homo sapiens
MGGSQSLQPAPASDLNLEASEAMSSDSEEAFETPESTTPVKAPPAPPPPPPEVIPEPEVSTQPPPEEPGCGSETVPVPDGPRSDSVEGSPFRPPSHSFSAVFDEDKPIASSGTYNLDFDNIELVDTFQTLEPRASDAKNQEGKVNTRRKSTDSVPISKSTLSRSLSLQASDFDGASSSGNPEAVALAPDAYSTGSSSASSTLKRTKKPRPPSLKKKQTTKKPTETPPVKETQQEPDEESLVPSGENLASETKTESAKTEGPSPALLEETPLEPAVGPKAACPLDSESAEGVVPPASGGGRVQNSPPVGRKTLPLTTAPEAGEVTPSDSGGQEDSPAKGLSVRLEFDYSEDKSSWDNQQENPPPTKKIGKKPVAKMPLRRPKMKKTPEKLDNTPASPPRSPAEPNDIPIAKGTYTFDIDKWDDPNFNPFSSTSKMQESPKLPQQSYNFDPDTCDESVDPFKTSSKTPSSPSKSPASFEIPASAMEANGVDGDGLNKPAKKKKTPLKTDTFRVKKSPKRSPLSDPPSQDPTPAATPETPPVISAVVHATDEEKLAVTNQKWTCMTVDLEADKQDYPQPSDLSTFVNETKFSSPTEELDYRNSYEIEYMEKIGSSLPQDDDAPKKQALYLMFDTSQESPVKSSPVRMSESPTPCSGSSFEETEALVNTAAKNQHPVPRGLAPNQESHLQVPEKSSQKELEAMGLGTPSEAIEITAPEGSFASADALLSRLAHPVSLCGALDYLEPDLAEKNPPLFAQKLQEELEFAIMRIEALKLARQIALASRSHQDAKREAAHPTDVSISKTALYSRIGTAEVEKPAGLLFQQPDLDSALQIARAEIITKEREVSEWKDKYEESRREVMEMRKIVAEYEKTIAQMIEDEQREKSVSHQTVQQLVLEKEQALADLNSVEKSLADLFRRYEKMKEVLEGFRKNEEVLKRCAQEYLSRVKKEEQRYQALKVHAEEKLDRANAEIAQVRGKAQQEQAAHQASLRKEQLRVDALERTLEQKNKEIEELTKICDELIAKMGKS

SEQ ID NO: 26 - protein, 838 aa, Homo sapiens
MSLQVLNDKNVSNEKNTENCDFLFSPPEVTGRSSVLRVSQKENVPPKNLAKAMKVTFQTPLRDPQTHRILSPSMASKLEAPFTQDDTLGLENSHPVWTQKENQQLIKEVDAKTTHGILQKPVEADTDLLGDASPAFGSGSSSESGPGALADLDCSSSSQSPGSSENQMVSPGKVSGSPEQAVEENLSSYSLDRRVTPASETLEDPCRTESQHKAETPHGAEEECKAETPHGAEEECRHGGVCAPAAVATSPPGAIPKEACGGAPLQGLPGEALGCPAGVGTPVPADGTQTLTCAHTSAPESTAPTNHLVAGRAMTLSPQEEVAAGQMASSSRSGPVKLEFDVSDGATSKRAPPPRRLGERSGLKPPLRKAAVRQQKAPQEVEEDDGRSGAGEDPPMPASRGSYHLDWDKMDDPNFIPFGGDTKSGCSEAQPPESPETRLGQPAAEQLHAGPATEEPGPCLSQQLHSASAEDTPVVQLAAETPTAESKERALNSASTSLPTSCPGSEPVPTHQQGQPALELKEESFRDPAEVLGTGAEVDYLEQFGTSSFKESALRKQSLYLKFDPLLRDSPGRPVPVATETSSMHGANETPSGRPREAKLVEFDFLGALDIPVPGPPPGVPAPGGPPLSTGPIVDLLQYSQKDLDAVVKATQEENRELRSRCEELHGKNLELGKIMDRFEEVVYQAMEEVQKQKELSKAEIQKVLKEKDQLTTDLNSMEKSFSDLFKRFEKQKEVIEGYRKNEESLKKCVEDYLARITQEGQRYQALKAHAEEKLQLANEEIAQVRSKAQAEALALQASLRKEQMRIQSLEKTVEQKTKENEELTRICDDLISKMEKI

SEQ ID NO: 27 - protein, 564 aa, Homo sapiens
MASTSTTIRSHSSSRRGFSANSARLPGVSRSGFSSVSVSRSRGSGGLGGACGGAGFGSRSLYGLGGSKRISIGGGSCAISGGYGSRAGGSYGFGGAGSGFGFGGGAGIGFGLGGGAGLAGGFGGPGFPVCPPGGIQEVTVNQSLLTPLNLQIDPTIQRVRAEEREQIKTLNNKFASFIDKVRFLEQQNKVLETKWTLLQEQGTKTVRQNLEPLFEQYINNLRRQLDSIVGERGRLDSELRGMQDLVEDFKNKYEDEINKRTAAENEFVTLKKDVDAAYMNKVELQAKADTLTDEINFLRALYDAELSQMQTHISDTSVVLSMDNNRNLDLDSIIAEVKAQYEEIAQRSRAEAESWYQTKYEELQVTAGRHGDDLRNTKQEIAEINRMIQRLRSEIDHVKKQCANLQAAIADAEQRGEMALKDAKNKLEGLEDALQKAKQDLARLLKEYQELMNVKLALDVEIATYRKLLEGEECRLNGEGVGQVNISVVQSTVSSGYGGASGVGSGLGLGGGSSYSYGSGLGVGGGFSSSSGRAIGGGLSSVGGGSSTIKYTTTSSSSRKSYKH

SEQ ID NO: 28 - protein, 430 aa, Homo sapiens
MSFTTRSTFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 29 - protein, 498 aa, Homo sapiens
MTVFLSFAFLAAILTHIGCSNQRRSPENSGRRYNRIQHGQCAYTFILPEHDGNCRESTTDQYNTNALQRDAPHVEPDFSSQKLQHLEHVMENYTQWLQKLENYIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGMFYTAGQNHGKLNGIKWHYFKGPSYSLRSTTMMIRPLDF

SEQ ID NO: 30 - protein, 344 aa, Homo sapiens
MAQKENSYPWPYGRQTAPSGLSTLPQRVLRKEPVTPSALVLMSRSNVQPTAAPGQKVMENSSGTPDILTRHFTIDDFEIGRPLGKGKFGNVYLAREKKSHFIVALKVLFKSQIEKEGVEHQLRREIEIQAHLHHPNILRLYNYFYDRRRIYLILEYAPRGELYKELQKSCTFDEQRTATIMEELADALMYCHGKKVIHRDIKPENLLLGLKGELKIADFGWSVHAPSLRRKTMCGTLDYLPPEMIEGRMHNEKVDLWCIGVLCYELLVGNPPFESASHNETYRRIVKVDLKFPASVPTGAQDLISKLLRHNPSERLPLAQVSAHPWVRANSRRVLPPSALQSVA

SEQ ID NO: 31 - protein, 655 aa, Homo sapiens
MSSSNVEVFIPVSQGNTNGFPATVSNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIEELGLDKVADSKVGTQFIRGVSGGERKRTSIGMELITDPSILSLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 32 - protein, 412 aa, Homo sapiens
MQPSSLLPLALCLLAAPASALVRIPLHKFTSIRRTMSEVGGSVEDLIAKGPVSKYSQAVPAVTEGPIPEVLKNYMDAQYYGEIGIGTPPQCFTVVFDTGSSNLWVPSIHCKLLDIACWIHHKYNSDKSSTYVKNGTSFDIHYGSGSLSGYLSQDTVSVPCQSASSASALGGVKVERQVFGEATKQPGITFIAAKFDGILGMAYPRISVNNVLPVFDNLMQQKLVDQNIFSFYLSRDPDAQPGGELMLGGTDSKYYKGSLSYLNVTRKAYWQVHLDQVEVASGLTLCKEGCEAIVDTGTSLMVGPVDEVRELQKAIGAVPLIQGEYMIPCEKVSTLPAITLKLGGKGYKLSPEDYTLKVSQAGKTLCLSGFMGMDIPPPSGPLWILGDVFIGRYYTVFDRDNNRVGFAEAARL

SEQ ID NO: 33 - protein, 750 aa, Homo sapiens
MGKSESQMDITDINTPKPKKKQRWTRLEISLSVLVLLLTIIAVRMIALYATYDDGICKSSDCIKSAARLIQNMDATTEPCRDFFKYACGGWLKRNVIPETSSRYGNFDILRDELEVVLKDVLQEPKTEDIVAVQKAKALYRSCINESAIDSRGGEPLLKLLPDIYGWPVATENWEQKYGASWTAEKAIAQLNSKYGKKVLINLFVGTDDKNSVNHVIHIDQPRLGLPSRDYYECTGIYKEACTAYVDFMISVARLIRQEERLPIDENQLALEMNKVMELEKEIANATAKPEDRNDPMLLYNKMRLAQIQNNFSLEINGKPFSWLNFTNEIMSTVNISITNEEDVVVYAPEYLTKLKPILTKYSARDLQNLMSWRFIMDLVSSLSRTYKESRNAFRKALYGTTSETATWRRCANYVNGNMENAVGRLYVEAAFAGESKHVVEDLIAQIREVFIQTLDDLTWMDAETKKRAEEKALAIKERIGYPDDIVSNDNKLNNEYLELNYKEDEYFENIIQNLKFSQSKQLKKLREKVDKDEWISGAAVVNAFYSSGRNQIVFPAGILQPPFFSAQQSNSLNYGGIGMVIGHEITHGFDDNGRNFNKDGDLVDWWTQQSASNFKEQSQCMVYQYGNFSWDLAGGQHLNGINTLGENIADNGGLGQAYRAYQNYIKKNGEEKLLPGLDLNHKQLFFLNFAQVWCGTYRPEYAVNSIKTDVHSPGNFRIIGTLQNSAEFSEAFHCRKNSYMNPEKKCRVW

SEQ ID NO: 34 - protein, 493 aa, Homo sapiens [sequence omitted]

SEQ ID NO: 35 - protein, 472 aa, Homo sapiens
MTTCSRQFTSSSSMKGSCGIGGGIGGGSSRISSVLAGGSCRAPSTYGGGLSVSSSRFSSGGAYGLGGGYGGGFSSSSSSFGSGFGGGYGGGLGAGLGGGFGGGFAGGDGLLVGSEKVTMQNLNDRLASYLDKVRALEEANADLEVKIRDWYQRQRPAEIKDYSPYFKTIEDLRNKILTATVDNANVLLQIDNARLAADDFRTKYETELNLRMSVEADINGLRRVLDELTLARADLEMQIESLKEELAYLKKNHEEEMNALRGQVGGDVNVEMDAAPGVDLSRILNEMRDQYEKMAEKNRKDAEEWFFTKTEELNREVATNSELVQSGKSEISELRRTMQNLEIELQSQLSMKASLENSLEETKGRYCMQLAQIQEMIGSVEEQLAQLRCEMEQQNQEYKILLDVKTRLEQEIATYRRLLEGEDAHLSSSQFSSGSQSSRDVTSSSRQIRTKVMDVHDGKVVSTHEQVLRTKN

SEQ ID NO: 36 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 37 - protein, 288 aa, Homo sapiens
MVGVGGGDVEDVTPRPGGCQISGRAARGCNGIPGAAAWEAALPRRRPRRHPSVNPRSRAAGSPRTRGRRTEERPSGSRLGDRGRGRALPGGRLGGRGRGRAPERVGGRGRGRGTAAPRAAPAARGSRPGPAGTMAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 38 - protein, 442 aa, Homo sapiens [sequence omitted]

SEQ ID NO: 39 - protein, 826 aa, Homo sapiens
MEGAGGANDKKKISSERRKEKSRDAARSRRSKESEVFYELAHQLPLPHNVSSHLDKASVMRLTISYLRVRKLLDAGDLDIEDDMKAQMNCFYLKALDGFVMVLTDDGDMIYISDNVNKYMGLTQFELTGHSVFDFTHPCDHEEMREMLTHRNGLVKKGKEQNTQRSFFLRMKCTLTSRGRTMNIKSATWKVLHCTGHIHVYDTNSNQPQCGYKKPPMTCLVLICEPIPHPSNIEIPLDSKTFLSRHSLDMKFSYCDERITELMGYEPEELLGRSIYEYYHALDSDHLTKTHHDMFTKGQVTTGQYRMLAKRGGYVWVETQATVIYNTKNSQPQCIVCVNYVVSGIIQHDLIFSLQQTECVLKPVESSDMKMTQLFTKVESEDTSSLFDKLKKEPDALTLLAPAAGDTIISLDFGSNDTETDDQQLEEVPLYNDVMLPSPNEKLQNINLAMSPLPTAETPKPLRSSADPALNQEVALKLEPNPESLELSFTMPQIQDQTPSPSDGSTRQSSPEPNSPSEYCFYVDSDMVNEFKLELVEKLFAEDTEAKNPFSTQDTDLDLEMLAPYIPMDDDFQLRSFDQLSPLESSSASPESASPQSTVTVFQQTQIQEPTANATTTTATTDELKTVTKDRMEDIKILIASPSPTHIHKETTSATSSPYRDTQSRTASPNRAGKGVIEQTEKSHPRSPNVLSVALSQRTTVPEEELNPKILALQNAQRKRKMEHDGSLFQAVGIGTLLQQPDDHAATTSLSWKRVKGCKSSEQNGMEQKTIILIPSDLACRLLGQSMDESGLPQLTSYDCEVNAPIQGSRNLLQGEELLRALDQVN

SEQ ID NO: 40 - protein, 707 aa, Homo sapiens
MSLWQPLVLVLLVLGCCFAAPRQRQSTLVLFPGDLRTNLTDRQLAEEYLYRYGYTRVAEMRGESKSLGPALLLLQKQLSLPETGELDSATLKAMRTPRCGVPDLGRFQTFEGDLKWHHHNITYWIQNYSEDLPRAVIDDAFARAFALWSAVTPLTFTRVYSRDADIVIQFGVAEHGDGYPFDGKDGLLAHAFPPGPGIQGDAHFDDDELWSLGKGVVVPTRFGNADGAACHFPFIFEGRSYSACTTDGRSDGLPWCSTTANYDTDDRFGFCPSERLYTQDGNADGKPCQFPFIFQGQSYSACTTDGRSDGYRWCATTANYDRDKLFGFCPTRADSTVMGGNSAGELCVFPFTFLGKEYSTCTSEGRGDGRLWCATTSNFDSDKKWGFCPDQGYSLFLVAAHEFGHALGLDHSSVPEALMYPMYRFTEGPPLHKDDVNGIRHLYGPRPEPEPRPPTTTTPQPTAPPTVCPTGPPTVHPSERPTAGPTGPPSAGPTGPPTAGPSTATTVPLSPVDDACNVNIFDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQCPED

SEQ ID NO: 41 - protein, 715 aa, Homo sapiens
MAANMYRVGDYVYFENSSSNPYLIRRIEELNKTANGNVEAKVVCFYRRRDISSTLIALADKHATLSVCYKAGPGADNGEEGEIEEEMENPEMVDLPEKLKHQLRHRELFLSRQLESLPATHIRGKCSVTLLNETESLKSYLEREDFFFYSLVYDPQQKTLLADKGEIRVGNRYQADITDLLKEGEEDGRDQSRLETQVWEAHNPLTDKQIDQFLVVARSVGTFARALDCSSSVRQPSLHMSAAAASRDITLFHAMDTLHKNIYDISKAISALVPQGGPVLCRDEMEEWSASEANLFEEALEKYGKDFTDIQQDFLPWKSLTSIIEYYYMWKTTDRYVQQKRLKAAEAESKLKQVYIPNYNKPNPNQISVNNVKAGVVNGTGAPGQSPGAGRACESCYTTQSYQWYSWGPPNMQCRLCASCWTYWKKYGGLKMPTRLDGERPGPNRSNMSPHGLPARSSGSPKFAMKTRQAFYLHTTKLTRIARRLCREILRPWHAARNPYLPINSAAIKAECTARLPEASQSPLVLKQAVRKPLEAVLRYLETHPRPPKPDPVKSVSSVLSSLTPAKVAPVINNGSPTILGKRSYEQHNGVDGNMKKRLLMPSRGLANHGQTRHMGPSRNLLLNGKSYPTKVRLIRGGSLPPVKRRRMNWIDAPGDVFYMPKEETRKIRKLLSSSETKRAARRPYKPIALRQSQALPPRPPPPAPVNDEPIVIED

SEQ ID NO: 42 - protein, 180 aa, Homo sapiens
CCEPRGSRARFGCWRLQPEFKPKQLEGTMANCERTFIAIKPDGVQRGLVGEIIKRFEQKGFRLVGLKFMQASEDLLKEHYVDLKDRPFFAGLVKYMHSGPVVAMVWEGLNVVKTGRVMLGETNPADSKPGTIRGDFCIQVGRNIIHGSDSVESAEKEIGLWFHPEELVDYTSCAQNWIYE

SEQ ID NO: 43 - protein, 640 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 44 - protein, 645 aa, Homo sapiens
MSERKEGRGKGKGKKKERGSGKKPESAAGSQSPALPPQLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCPNEFTGDRCQNYVMASFYKHLGIEFMEAEELYQKRVLTITGICIALLVVGIMCVVAYCKTKKQRKKLHDRLRQSLRSERNNMMNIANGPHHPNPPPENVQLVNQYVSKNVISSEHIVEREAETSFSTSHYTSTAHHSTTVTQTPSHSWSNGHTESILSESHSVIVMSSVENSRHSSPTGGPRGRLNGTGGPRECNSFLRHARETPDSYRDSPHSERYVSAMTTPARMSPVDFHTPSSPKSPPSEMSPPVSSMTVSMPSMAVSPFMEEERPLLLVTPPRLREKKFDHHPQQFSSFHHNPAHDSNSLPASPLRIVEDEEYETTQEYEPAQEPVKKLANSRRAKRTKPNGHIANRLEVDSNTSSQSSNSESETEDERVGEDTPFLGIQNPLAASLEATPAFRLADSRTNPAGRFSTQEEIQARLSSVIANQDPIAV

SEQ ID NO: 45 - protein, 198 aa, Homo sapiens
MSNVRVSNGSPSLERMDARQAEHPKPSACRNLFGPVDHEELTRDLEKHCRDMEEASQRKWNFDFQNHKPLEGKYEWQEVEKGSLPEFYYRPPRPPKGACKVPAQESQDVSGSRPAAPLIGAPANSEDTHLVDPKTDPSDSQTGLAEQCAGIRKRPATDDSSTQNKRANRTEENVSDGSPNAGSVEQTPKKPGLRRRQT

SEQ ID NO: 46 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETPVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 47 - protein, 395 aa, Homo sapiens
MRALLARLLLCVLVVSDSKGSNELHQVPYKSKTCYEGNGHFYRGKASTDTMGRPCLPWNSATVLQQTYHAHRSDALQLGLGKHNYCRNPDNRRRPWCYVQVGLKLLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 48 - protein, 94 aa, Homo sapiens
MGSELETAMETLINVFHAHSGKEGDKYKLSKKELKELLQTELSGFLDAQKDVDAVDKVMKELDENGDGEVDFQEYVVLVAALTVACNNFFWENS

SEQ ID NO: 49 - protein, 1630 aa, Homo sapiens
MLKCIPLWRCNRHVESVDKRHCSLQAVPEEIYRYSRSLEELLLDANQLRELPKPFFRLLNLRKLGLSDNEIQRLPPEVANFMQLVELDVSRNDIPEIPESIKFCKALEIADFSGNPLSRLPDGFTQLRSLAHLALNDVSLQALPGDVGNLANLVTLELRENLLKSLPASLSFLVKLEQLDLGGNDLEVLPDTLGALPNLRELWLDRNQLSALPPELGNLRRLVCLDVSENRLEELPAELGGLVLLTDLLLSQNLLRRLPDGIGQLKQLSILKVDQNRLCEVTEAIGDCENLSELILTENLLMALPRSLGKLTKLTNLNVDRNHLEALPPEIGGCVALSVLSLRDNRLAVLPPELAHTTELHVLDVAGNRLQSLPFALTHLNLKALWLAENQAQPMLRFQTEDDARTGEKVLTCYLLPQQPPLSLEDAGQQGSLSETWSDAPPSRVSVIQFLEAPIGDEDAEEAAAEKRGLQRRATPHPSELKVMKRSIEGRRSEACPCQPDSGSPLPAEEEKRLSAESGLSEDSRPSASTVSEAEPEGPSAEAQGGSQQEATTAGGEEDAEEDYQEPTVHFAEDALLPGDDREIEEGQPEAPWTLPGGRQRLIRKDTPHYKKHFKISKLPQPEAVVALLQGMQPDGEGPVAPGGWHNGPHAPWAPRAQKEEEEEEEGSPQEEEEEEEEENRAEEEEASTEEEDKEGAVVSAPSVKGVSFDQANNLLIEPARIEEEELTLTILRQTGGLGISIAGGKGSTPYKGDDEGIFISRVSEEGPAARAGVRVGDKLLEVNGVALQGAEHHEAVEALRGAGTAVQMRVWRERMVEPENAVTITPLRPEDDYSPRERRGGGLRLPLLPPESPGPLRQRHVACLARSERGLGFSIAGGKGSTPYRAGDAGIFVSRIAEGGAAHRAGTLQVGDRVLSINGVDVTEARHDHAVSLLTAASPTIALLLEREAGGPLPPSPLPHSSPPTAAVATTSITTATPGVPGLPSLAPSLLAAALEGPYPVEEIRLPRAGGPLGLSIVGGSDHSSHPFGVQEPGVFISKVLPRGLAARSGLRVGDRILAVNGQDVRDATHQEAVSALLRPCLELSLLVRRDPAPPGLRELCIQKAPGERLGISIRGGARGHAGNPRDPTDEGIFISKVSPTGAAGRDGRLRVGLRLLEVNQQSLLGLTHGEAVQLLRSVGDTLTVLVCDGFEASTDAALEVSPGVIANPFAAGIGHRNSLESISSIDRELSPEGPGKEKELPGQTLHWGPEATEAAGRGLQPLKLDYRALAAVPSAGSVQRVPSGAAGGKMAESPCSPSGQQPPSPPSPDELPANVKQAYRAFAAVPTSHPPEDAPAQPPTPGPAASPEQLSFRERQKYFELEVRVPQAEGPPKRVSLVGADDLRKMQEEEARKLQQKRAQMLREAAEAGAEARLALDGETLGEEEQEDEQPPWASPSPTSRQSPASPPPLGGGAPVRTAKAERRHQERLRVQSPEPPAPERALSPAKLRALEAEKRALWRAARMKSLEQDALRAQMVLSRSQEGRGTRGPLERLAEAPSPAPTPSPTPVEDLGPQTSTSPGRLSPDFAEELRSLEPSPSPGPQEEDGEVALVLLGRPSPGAVGPEDVALCSSRRPVRPGRRGLGPVPS

SEQ ID NO: 50 - protein, 377 aa, Homo sapiens
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 51 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 52 - protein, 207 aa, Homo sapiens
MAPFEPLASGILLLLWLIAPSRACTCVPPHPQTAFCNSDLVIRAKFVGTPEVNQTTLYQRYEIKMTKMYKGFQALGDAADIRFVYTPAMESVCGYFHRSHNRSEEFLIAGKLQDGLLHITTCSFVAPWNSLSLAQRRGFTKTYTVGCEECTVFPCLSIPCKLQSGTHCLWTDQLLQGSEKGFQSRHLACLPREPGLCTWQSLRSQIA

SEQ ID NO: 53 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 54 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

(19) United States
(12) Patent Application Publication
(10) Pub. No.: US 2005/0221398 A1
(43) Pub. Date: Oct. 6, 2005

(54) Protein expression profiling and breast cancer prognosis

(75) Inventors: Jocelyne Jacquemier, Marseille (FR); Francois Bertucci, Marseille (FR); Daniel Birnbaum, Marseille (FR); Stephane Debono, Marseille (FR); Rebecca Tagett, Marseille (FR)

Correspondence Address: IP GROUP OF DLA PIPER RUDNICK GRAY CARY US LLP, 1650 MARKET ST, SUITE 4900, PHILADELPHIA, PA, 19103, US

(73) Assignees: Ipsogen, SAS, a corporation of France, Marseille (FR); INSERM, a corporation of France, Paris (FR); Institut Paoli-Calmettes, a corporation of France, Marseille (FR)

(21) Appl. No.: 11/037,713

(22) Filed: Jan. 18, 2005

Related U.S. Application Data
(60) Provisional application No. 60/537,412, filed on Jan. 16, 2004.

(51) Int. Cl. C12Q 1/68; G01N 33/574; G01N 33/00

(52) U.S. Cl. 435/7.23; 436/86

(57) ABSTRACT

A method for analyzing differential protein expression associated with histopathologic features of breast disease including detecting overexpression or underexpression of a pool of proteins in breast tissues or cells, the pool including at lease one of a protein set including Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2, TACC3, Cytokeratin 6, Cytokeratin 18, Ang1, AuroraB, BCRP1, CathepsinD, CD10, CD44, CK14, Cox2, FGF2, GATA4, Hif1a, MMP9, MTA1, NM23, NRG1a, NRG1beta, P27, Parkin, PLAU, S100, SCRIBBLE, Smooth Muscle Actin, THBS1 and TIMP1.

### RELATED APPLICATION

This patent application claims priority of U.S. Provisional Application No. 60/537,412, filed Jan. 16, 2004. This earlier provisional application is hereby incorporated by reference.

### FIELD OF THE INVENTION

This invention relates to protein analysis and, in particular, to protein expression profiling of breast tumors and cancers.

### BACKGROUND

Adjuvant systemic therapy has a favorable impact on survival in patients with early breast cancer.^{1, 2} The decision to give or withhold such therapy is based upon a series of histoclinical prognostic criteria reviewed in consensus conferences, i.e., National Institute Health NIH and St-Gallen.^{3, 4} However, despite the establishment of standardized criteria, the heterogeneity of breast tumors remains poorly understood. For example, clinical treatment decisions on whether to treat patients with node-negative breast cancer by surgery and radiotherapy alone, or in combination with adjuvant chemotherapy are currently being made with scant information on patient risk for metastatic relapse. Additionally, identifying among the patients who receive chemotherapy those who will benefit and those who will not benefit from standard anthracyclin-based protocols remains elusive. However, the relatively limited efficacy of current protocols (about 30-40% of failure rate) and the increasing availability of new therapies make this issue clinically important. Furthermore, the development of molecularly-targeted drugs such as trastuzumab (Herceptin™), a monoclonal antibody against the ERBB2 tyrosine kinase receptor, is needed.⁵ With few exceptions, such as estrogen receptor and ERBB2 receptor, the available molecular markers are of limited value in clinical practice.

High-throughput molecular technologies such as DNA arrays, have recently significantly contributed to enhance understanding of the molecular complexity of breast cancer.⁶ Several studies have demonstrated the potential clinical utility of gene expression signatures defined by the combined RNA expression of a few tens of genes. These signatures have lead to the development of a new molecular taxonomy of disease, including the identification of previously indistinguishable prognostic subclasses.⁷⁻¹⁵ The clinical impact of these tests on disease management must be subsequently evaluated in large retrospective and prospective studies of adequate statistical power on fully annotated patient samples, followed by the development of gene expression-based diagnostics adapted to the clinical setting.

Unfortunately, the cost, technical complexity, and interpretation of DNA microarray technology still complicate investigation with cancer specimens and are currently unsuitable for routine use in the standard clinical setting. Issues that must be addressed prior to validation and integration of this technology to clinical pathology laboratories include the requirement for high-quality RNA extracted from unfixed tissues, intra-tumoral heterogeneity of excised patient samples, and bias resulting from the asymmetry of variables with a number of hybridized samples greatly inferior to the number of genes being tested leading to non-trivial statistical problems. Finally, the sensitivity, specificity, reproducibility and technical feasibility outside large academic centers will have to be addressed, and experimental conditions will have to be standardized and data compared in multi-center clinical trials.

Additional opportunities to validate and/or identify prognostic expression signatures are provided by alternative high-throughput approaches, which may be used either separately or in combination with DNA microarrays. One of these is the tissue microarray (TMA) technique,¹⁶⁻¹⁸ which allows for the simultaneous study of hundreds of tumor specimens at the DNA, RNA or protein level. Immunohistochemistry (IHC) is applicable to paraffin-embedded samples that constitute the bulk of pathology archives, avoiding the requirement for high-quality RNA extracted from frozen specimens. IHC is relatively inexpensive, straightforward and well established in standard clinical pathology laboratories. Thus, IHC on TMA may be a practical approach both in validation studies and in routine testing. However, analytical classification methods to efficiently process and interpret multiple target IHC data have not been previously developed.

Recent studies have shown the reliability of hierarchical clustering for classifying cancers when applied to IHC TMA data of a significant range of markers.¹⁹⁻²⁴ However, none addressed the prognostic issue.

### SUMMARY OF THE INVENTION

This invention in a broad sense provides a means of analyzing histopathologic features of breast disease, in particular, of classifying breast cancers into prognostically relevant subclasses. After exhaustive testing on a retrospective panel of 552 early breast cancer samples we found that this classification was possible by analyzing a consistent set of proteins. Classification of samples, based on this multidimensional protein data set, was first done using classical unsupervised hierarchical clustering. We then developed a supervised bioinformatic method that further improved the classification as compared with usual prognostic factors.

The invention provides a protein expression signature identified by protein expression profiling which may be used for analyzing histopathologic features of breast disease as well as methods for carrying out such analysis. In particular, protein expression profiling is a clinically useful approach to assess breast cancer heterogeneity and prognosis in patients with stage I, II, or III disease. It may be used both for breast tumor management in clinical settings and as a research tool in academic laboratories.

The invention provides in one aspect a method for analyzing differential protein expression associated with histopathologic features of breast disease, in particular, breast tumours, e.g., breast carcinomas, comprising detecting overexpression or underexpression of a pool of proteins in breast tissues or cells, the pool comprising all or part of a protein set comprising:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2, TACC3.

By “all or part” is meant 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or 52 proteins.

By “Cytokeratin 5/6” is meant Cytokeratin 5 and/or Cytokeratin 6. The same is applicable to “Cytokeratin 8/18.”

The following table displays proteins of the invention and their corresponding amino-acid sequences (SEQ ID NO. 1 to 52). These proteins are identified by their common names (first column) in the methods, libraries, sets, pools, etc. of the invention. Other names in the literature which designate the same proteins (alias, synonyms, etc.) are included and are incorporated herein by reference.

The invention may also define these proteins by their amino-acid (polypeptidic) sequences (SEQ ID NO.), or portions or modifications thereof in accordance with the definition of “protein” provided in Table 1 below.

TABLE 1

| Protein Name | SEQ ID NO. |
|---|---|
| Afadin | 1 |
| Aurora A | 2 |
| a-Catenin | 3 |
| b-Catenin | 4 |
| BCL2 | 5 |
| Cyclin D1 | 6 |
| Cyclin E | 7 |
| Cytokeratin 5 | 8 |
| Cytokeratin 8 | 9 |
| E-Cadherin | 10 |
| EGFR | 11 |
| ERBB2 | 12 |
| ERBB3 | 13 |
| ERBB4 | 14 |
| Estrogen receptor | 15 |
| FGFR1 | 16 |
| FHIT | 17 |
| GATA3 | 18 |
| Ki67 | 19 |
| Mucin 1 | 20 |
| P53 | 21 |
| P-Cadherin | 22 |
| Progesterone receptor | 23 |
| TACC1 | 24 |
| TACC2 | 25 |
| TACC3 | 26 |
| Cytokeratin 6 | 27 |
| Cytokeratin 18 | 28 |
| Ang1 | 29 |
| AuroraB | 30 |
| BCRP1 | 31 |
| CathepsinD | 32 |
| CD10 | 33 |
| CD44 | 34 |
| CK14 | 35 |
| Cox2 | 36 |
| FGF2 | 37 |
| GATA4 | 38 |
| Hifla | 39 |
| MMP9 | 40 |
| MTA1 | 41 |
| NM23 | 42 |
| NRG1a | 43 |
| NRG1beta | 44 |
| P27 | 45 |
| Parkin | 46 |
| PLAU | 47 |
| S100 | 48 |
| SCRIBBLE | 49 |
| Smooth Muscle Actin | 50 |
| THBS1 | 51 |
| TIMP1 | 52 |
| VEGFc | 53 |
| Vimentine | 54 |

“Over or underexpression of a pool of protein” means that overexpression of certain proteins are detected simultaneously to the underexpression of others the proteins. “Simultaneously” means concurrent with or within a biologic or functionally relevant period of time during which the over expression of a protein may be followed by the under expression of another protein, or conversely, e.g., because both expressions are directly or indirectly correlated.

In a further aspect, the invention provides a method for analyzing differential protein expression associated with histopathologic features of breast disease comprising detecting overexpression or underexpression of a pool of protein in breast tissues comprising a protein set comprising:
Aurora A, a-Catenin, b-Catenin, Cyclin D1, Cytokeratin 8/18, ERBB2, ERBB3, Estrogen receptor, FGFR1, Ki67, Mucin 1, P53, P-Cadherin, Progesterone receptor and TACC2.

In a further aspect, the invention provides a method for analyzing differential protein expression associated with histopathologic features of breast disease comprising detecting overexpression or underexpression of a pool of protein in breast tissues comprising a protein set comprising:
Afadin, Aurora A, a-Catenin, BCL2, Cyclin D1, Cytokeratin 5/6, Cytokeratin 8/18, E-Cadherin, ERBB2, ERBB3, ERBB4, Estrogen receptor, FGFR1, FHIT, Ki67, Mucin 1, P53, P-Cadherin, Progesterone receptor, TACC2 and TACC3.

According to a preferred aspect, the pool of protein comprises a protein set comprising:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2 and TACC3.

According to another aspect, the pool of protein comprises a protein set comprising all proteins of the Table 1 above.

The method further comprises at least one of the following aspects:
detecting of overexpression of at least one, preferably at least two, three or all of the following proteins:
   EGFR, P53, Ki67, FGFR1, ERBB2, ERBB3, ERBB4, Cyclin D1, Cyclin E and Cytokeratin 5/6.
detecting overexpression of at least one, preferably at least two, three or all of the following proteins:
   Estrogen Receptor, FHIT, GATA3, Mucin 1, P-Cadherin, Progesterone receptor, TACC1, TACC2, TACC3, Afadin, Aurora A, α-Catenin, β-Catenin, BCL2, Cytokeratin 8/18 and E-Cadherin.

The method may further comprise at least one of the following aspects:
detecting of overexpression of at least one, preferably at least two, three or all of the following proteins:
   BCRP1, CK14, GATA4, NRG1a, NRG1beta, S100, SCRIBBLE, Smoth Muscle Actin and CD44.
detecting of underexpression of at least one, preferably at least two, three or all of the following proteins:
   Ang1, AuroraB, CathepsinD, THBS1, TIMP1, NM23, MMP9, MTA1, P27, VEGFc and Vimentine.

A further aspect of the invention provides a protein library useful for molecular characterization of histopathologic features of breast disease comprising or corresponding to a pool of protein sequences, over or under expressed, in breast tissue or cells, the pool corresponding to the protein sets previously described.

Preferably, the protein librairies may be immobilized on a solid support which may preferably be selected from the group comprising nylon membrane, nitrocellulose membrane, polyvinylidene difluoride, glass slide, glass beads, polystyrene plates, membranes on glass support and silicon chip or gold chip.

In a further aspect, the invention provides a method for analyzing differential protein expression associated with histopathologic features of breast disease comprising detecting overexpression or underexpression of a pool of protein in breast tissues comprising:
a) obtaining breast tissue cells from a patient, and
b) measuring in the tissue cells obtained in step (a) over or underexpression of proteins of a library as previously described.

Alternatively to breast tissue cells from a patient, detecting over or under expression of the pool of protein may be carried out on breast tumor cell lines.

The proteins may be directly or indirectly labeled before reaction step (b) with a label which may be selected from the group comprising radioactive, colorimetric, enzymatic, molecular amplification, bioluminescent or fluorescent labels. Advantageously, one or more specific label are used for each protein of the library. A person skilled the art will be able to select appropriate labels and labelling methods to carry out the invention. For example, one may use a label selected in the group comprising, but not limited to: biotine and digoxygenin.

Measuring over or under expression of proteins may be carried out on cell or tissue, frozen or embedded in any appropriate material, e.g., paraffin, e.g. tissue microarray. Various known methods may be used sicj as, e.g., ImmunoHistoChemistry (IHC) technologies. Measuring over or under expression of proteins may be also be carried out with, e.g., protein (micro)arrays, antibody (micro)arrays, antigen (micro)arrays or any other appropriate technology, e.g., by using the previously defined supports.

According to an advantageous aspect, the method for analysing differential protein expression of the invention further comprises:
a) obtaining a control sample;
b) measuring in the control sample obtained in step (a) expression level of each protein corresponding to the library; and
c) comparing expression level of each protein with the level of equivalent protein in breast tissue cells from a patient, or in cell lines.

The invention is useful for detecting, diagnosing, staging, monitoring, predicting, preventing conditions associated with breast cancer. It is particularly useful for predicting clinical outcome of breast cancer and/or predicting occurrence of metastatic relapse and/or determining the stage or aggressiveness of a breast disease in at least about 50%, e.g., at least about 55%, e.g., at least about 60%, e.g., at least about 65%, e.g., at least about 70%, e.g., at least about 75%, e.g., at least about 80%, e.g., at least about 85%, e.g., at least about 90%, e.g., at least about 95%, e.g., about 100% of the patients. The invention is also useful for selecting more appropriate doses and/or schedule of chemotherapeutics and/or biopharmaceuticals and/or radiation therapy to circumvent toxicities in a patient.

The invention is also useful for selecting appropriate doses and/or schedule of chemotherapeutics and/or (bio)pharmaceuticals, and/or targeted agents, among which include Aromatase Inhibitors (e.g., Exomestane, Anastrazole, Letrozole), Anti-estrogens (e.g., Fluvestrant, Tamoxifen), Taxanes (e.g., PacliTaxol, Docetaxel), Antracyclines (e.g., Doxurubicin, Cyclophosphamide), CHOP (Doxurubicin, Cyclophosphamide, ocovorin, prednisone when taken in combination). Other drugs such as Velcade™, 5-Fluorouracil, Vinblastine, Gemcitabine, Methotrexate, Goserelin, Irinotecan, Thiotepa, Topotecan or Toremifene are included as well.

Targeted therapies include use of Iressa (gefitnib, ZD1839, anti-EGFR, PDGFR, c-kit, Astra-Zeneca); ABX-EGFR (anti-EGFR, Abgenix/Amgen); Zamestra (FTI, J & J/Ortho-Biotech); Herceptin (anti-HER2/neu, Genentech); Avastin (bevancizumab, anti-VEGF antibody, Genentech); Tarceva (ertolinib, OSI-774, RTK inhibitor, Genentech-Roche); ZD66474 (anti-VEGFR, Astra-Zeneca); Erbitux (IMC-225, cetuximab, anti-EGFR, Imclone/BMS); Oncolar (anti-GRH, Novartis); PD-183805 (RTK inhibitor, Pfizer); EMD72000, (anti-EGFR/VEGF ab, MerckKgaA); CI-1033 (HER2/neu & EGF-R dual inhibitor, Pfizer); EGF10004; Herzyme (anti-HER2 ab, Medizyme Pharmaceuticals); Corixa (Microsphere delivery of HER2/neu vaccine, Medarex).

Further relevant anti-breast cancer agents are described by Awada et al. in “The pipeline of new anticancer agents for breast cancer treatment in 2003,” Critical Reviews in Oncology/Hematology 48 (2003) 45-63, the content of which is incorporated herein by reference.

Advantageously, in the method, breast tissue cell may be obtained from a patient regardless of whether the patient has received or not a neo-adjuvant or adjuvant, e.g., systemic, therapy. Similarly, treated or untreated cell lines may be used.

Advantageously, in the method, breast tissue cell may be obtained from a patient regardless of ER receptor expression.

In a further aspect, the invention provides a method for treating a patient with breast cancer comprising (i) implementing a method for analysing differential protein expression on a sample from the patient, and (ii) determining a treatment for the patient based on the analysis of differential protein expression profile obtained in step i).

In a further aspect, the invention relates to a method for analyzing differential protein expression associated with histopathologic features of breast disease, wherein detecting overexpression or underexpression of the pool of protein in breast tissues comprises detecting overexpression or underexpression of nucleic acids coding for the proteins.

The invention further relates to a nucleic acids library useful for the molecular characterization of histopathologic features of breast disease comprising nucelic acids coding for the over or underexpressed proteins, or equivalents thereof.

The sequences of the nucleic acids of the library are easily available for a person skilled in the art that may, for example, use printed publications describing the sequences and/or public databases, e.g., the National Center for Biotechnological Information (NCBI) database, that provide such sequences as well. The content of the NCBI database may be available via internet at the following adress http://www.ncbi.nlm.nih.gov/.

### BRIEF DESCRIPTION OF THE DRAWINGS

FIG. 1 shows hierarchical clustering analysis of global protein expression profiles in breast cancer as measured by IHC on TMA. FIG. 1A: Graphical representation of hierarchical clustering results based on expression profiles of 26 proteins in 552 early breast cancer samples. Each row represents a sample and each column represents a protein. Immunostaining results are depicted according to a color scale: red or brown for strong or moderate positive staining, respectively, green for negative staining, gray for missing data. Dendrograms of samples (to the left of matrix) and proteins (above matrix) represent overall similarities in expression profiles. Three major clusters of tumors (A1, A2 and B) are shown (A1 and A2 correspond to luminal cells; B corresponds to basal cells). Colored bars to the right and colored branches in the dendrogram indicate the locations of 3 sample clusters of interest zoomed in C. FIG. 1B: Dendrogram of proteins. Two major clusters “P1” (basal/stem cells) and “P2” (luminal/glandular cells) are identified and further divided in 4 smaller clusters designated “proliferation”, “mitosis”, “ER-related” and “adhesion” cluster, respectively. FIG. 1C: Expanded view of selected sample clusters showing a partial grouping of tumors with similar histological type (LOB: lobular, DUC: ductal, OTH: other, MIX: mixed; blue bar) or ER status (positive, red bar and negative, orange bar).

FIG. 2 shows classification of 552 breast cancer samples based on the expression of the 21-protein discriminator set identified by supervised analysis. FIGS. 2A and 2B: Correlations between the molecular grouping based on the combined expression of the 21 proteins and the occurrence of metastatic relapse in the learning (A) and the validation (B) set of samples. FIG. 2C: Supervised classification of all 552 samples using the 21-protein expression signature. Each row of the data matrix (left panel) represents a sample and each column represents a protein. Immunostaining results are depicted according to the color scale used in FIG. 1. The 21 proteins, listed above the matrix (ER*: means of three independent ER analyses), are ordered from left to right according to decreasing ΔP (ΔP is the difference between the probability of positive staining and the probability of negative staining in non-metastatic samples). Tumor samples are numbered from 1 to 552 and are ordered from top to bottom according to their increasing “Metastasis Score” (right panel). The orange dashed line indicates the threshold 0 that separates the two classes of samples, “poor-prognosis” (under the line) and “good-prognosis” (above the line). The middle panel indicates the occurrence (black square) or not (white square) of metastatic relapse for each patient.

FIG. 3 shows a Kaplan-Meier analysis of the metastasis-free survival of patients with breast cancer according to the molecular classification based on the 21-protein expression signature or the St-Gallen and the NIH consensus criteria. Patients (pts) were classified in the “good-prognosis” class or the “poor-prognosis” class using the 21-protein signature identified by supervised analysis (A, B, E and F) or in the “low risk” class or the “high risk” class using the St-Gallen and the NIH consensus criteria (C and D). The P-values are calculated using the log-rank test. FIG. 3A: Survival of all 552 patients. FIG. 3B: Survival of 292 patients with node-negative cancer (N−) and 255 patients with node-positive cancer (N+). The difference of survival is significant between the “good-prognosis” class and the “poor-prognosis” class for the node-negative patients, as well as for the node-positive patients. In contrast, survival is not significantly different between the node-positive patients from the “good-prognosis class” and the node-negative patients from the “poor-prognosis class”. FIG. 3C: Survival of 292 patients with node-negative cancer (N−) according to the St-Gallen criteria. FIG. 3D: Survival of 292 patients with node-negative cancer (N−) according to the NIH criteria. FIG. 3E: Survival of 186 patients without any adjuvant chemotherapy (CT) and hormone therapy (HT). FIG. 3F: Survival of 133 patients who received adjuvant chemotherapy (CT) without hormone therapy (HT).

FIG. 4 shows expression of proteins studied by IHC on tissue microarrays (TMA). FIG. 4A: Representative Hematoxylin-Eosin and Safran staining of a paraffin block section (25×30 mm²) from a TMA containing 552 early breast cancer cases with 0.6 mm tumor cores. FIG. 4B: Immunohistochemical staining of a tumor core for the 21 proteins identified by supervised analysis (magnification ×200). FIG. 4C: Examples of IHC staining for 5 proteins with differential expression in cancer tissue (bottom) compared with normal tissue (top). 1, FHIT expression in cytoplasm in normal lobules, down-regulation in cancer sample (arrow); 2, Apical normal expression of MUC1, down-regulation and miss-localization in the cytoplasm of cancer sample (arrow); 3, Absence of ERBB2 expression in normal lobule (arrow), overexpression on the cytoplasmic membrane in positive cancer sample (arrow); 4, Absence of nuclear expression of Cyclin D1 in normal lobules (arrow), overexpression in nucleus of positive cancer sample (arrow); 5, Normal myoepithelial cells are immunostained by P Cadherin (arrow), overexpression in cancer sample (arrow). Magnification is ×400.

### DETAILED DESCRIPTION

Definitions

“Aggressiveness of cancer” refers to cancer growth rate or potential to metastasize; a so-called “aggressive cancer” will grow or metastasize rapidly or significantly affect overall health status and quality of life.

“Adjuvant therapy” refers to treatment involving radiation, chemotherapy (drug treatment), biologic therapy (vaccines) or hormone therapy, or any combination given after primary treatment.

“Antibody” is intended to include whole antibodies, e.g., of any isotype, and includes fragments thereof which are also specifically reactive with a vertebrate, e.g., mammalian, protein. Antibodies can be fragmented using conventional techniques and the fragments screened for utility in the same manner as described above for whole antibodies. Thus, the term includes segments generated by proteolytic cleavage or prepared recombinant portions of an antibody molecule capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. Antibodies may include polyclonal, monoclonal, or other purified preparations of antibodies and recombinant antibodies.

“Associated with” refers to a disease in a subject which is caused by, contributed to by, or causative of an abnormal level of expression of a protein.

“Control” comprises, for example, proteins from a sample of the same patient or from a pool of different patients, or selected among reference proteins which may be already known to be over or under expressed. The expression level of the control can be an average or an absolute value of the expression of reference proteins. These values may be processed to accentuate the difference relative to the expression of the proteins according to the invention. The analysis of the over or under expression of proteins can be carried out on samples such as biological material derived from any mammalian cells, including cell lines, xenografts, human tissues preferably breast tissue and the like. The method according to the invention may be performed on sample from a, e.g., cell lines, healthy donors, patients or an animal (for example for veterinary application or preclinical studies).

“Directly or indirectly labeled” include proteins the sub-constituants of which, i.e., amino acids or amino acid groups or atoms, are themselves labeled (directly), as well as proteins labeled by the intermediate of any element able to recognize and bind to the targeted protein, e.g., an antibody.

“Equivalent” includes nucleic acids encoding functionally equivalent proteins. Equivalent nucleotide sequences include sequences that differ by one or more nucleotide substitutions, additions or deletions, such as allelic variants and, will, therefore, include sequences that differ from the nucleotide sequence of the nucleic acids of the invention because of the degeneracy of the genetic code.

“Good-prognosis” and “poor-prognosis,” respectively, refer to favorable (e.g., remission) or unfavorable (e.g., metastasis, death) patient clinical outcome.

“Histopathologic features of breast diseases” includes diseases, disorders or conditions known as, lethally or not, affecting breast cells and/or tissues, including but not limited to breast tumours, for example i) non cancerous breast diseases, for example, hyperplasias, metaplasias, fibroadenomas, fibrocystic disease, papillomas, sclerosing adenosis or preneoplastic, or ii) breast cancer. “Breast cancer” includes but is not limited to:
A) noninvasive breast cancers including i) ductal carcinoma in situ (also called “intraductal carcinoma” or DCIS), consisting of cancer cells in the lining of the duct, ii) Lobular carcinoma in situ, or LCIS (also known as “lobular neoplasia”);
B) Invasive cancer occurring when cancer cells spread beyond the basement membrane which covers the underlying connective tissue in the breast, and which include i) Infiltrating ductal carcinoma that penetrates the wall of a duct, and ii) Infiltrating lobular carcinoma which spreads through the wall of a lobule and may sometimes appear in both breasts, sometimes in several separate locations.

“ImmunoHistoChemistry (IHC)” refers to methods using histochemical localization of immunoreactive substances using antibodies as reagents on cells or tissues by technologies such as, but not limited to flow cytometry, ELISA, Western and Southwestern Blot Analysis, and frozen and paraffin-embedded samples.

“Nucleic acids” refers to polynucleotides, e.g., isolated, such as deoxyribonucleic acid (DNA), and, where appropriate, ribonucleic acid (RNA). The term should also be understood to include, as equivalents, analogs of RNA or DNA made from nucleotide analogs, and, as applicable to the aspect being described, single (sense or antisense) and double-stranded polynucleotides. ESTs, chromosomes, cDNAs, mRNAs, and rRNAs are representative examples of molecules that may be referred to as nucleic acids.

“Over or underexpression” may comprise the detection of differences in expression of the proteins according to the invention in relation to at least one control.

“Predicting clinical outcome” refers to the ability for one skilled in the art to classify patients into at least two classes “good prognosis” and “bad prognosis” showing significantly different long-term Metastasis Free Survival (MFS).

“Protein” refers to a polypeptide with a primary, secondary, tertiary or quaternary structure, or any portion or modification, e.g., a mutant, or isoform thereof. A “portion” or “modification” of a protein retains at least one biological or antigenic characteristic of a native (wild-type) protein.

“Protein microarray” refers to a spatially defined and separated collection of individual proteins immobilised on a solid surface.

“Treating” as used herein is intended to encompass treating as well as ameliorating at least one symptom of the condition or disease.

We combined IHC and TMA to measure the expression levels of selected proteins in a consecutive series of 552 patients with early stage breast cancer. We determined protein combinations to refine tumor classification and improve the prognostic classification of disease.

Protein Expression Profiling Identifies Subclasses of Breast Cancer

Analysis and interpretation of the large amount of data generated (552 samples and 26 antibodies, about 14,000 data points) caused us to develop bioinformatic tools. As a first step, we applied pre-existing unsupervised hierarchical clustering algorithms as previously reported.¹⁹⁻²⁴ Two recent studies on breast cancer analyzed the expression of 15 proteins in 166 tumors,²² and 13 proteins on 107 samples,¹⁹ respectively. Several of these markers were included in this work (BCL2, ER, PR, ERBB2, EGFR, Cyclins, Cytokeratins, MIB1, P53), allowing for direct comparison of results. In our analysis, clustering allowed the identification of four major coherent protein clusters designated according to the function of most included proteins: “ER-related cluster”, “adhesion cluster”, “mitosis cluster” and “proliferation cluster.” Correlated expression of proteins may be due to different mechanisms such as coregulation (e.g., ER/BCL2³⁰), functional interaction (e.g., STK6/Taxins^{27, 28}), phenotypic association (e.g., ERBB2/P53³¹) or chromosomal location (e.g., FGFR1/TACC1 located on 8p11). Some co-expressed proteins were previously reported in RNA or protein expression profiling studies. For example, ER, PR, BCL2 and GATA3 clustered together.^{8-10, 13} This “ER-related cluster” was negatively correlated with the “mitosis” and “proliferation” clusters, in agreement with the higher proliferation index in ER-negative tumors³² and the known proliferation-differentiation balance in carcinomas. The “ER-related cluster” was close to the “adhesion cluster” that included other markers that may correlate positively with ER expression such as FHIT,³³ CK8/18,^{19, 22} CCND1³⁴ and MUC1.⁸ Our “proliferation cluster” had some similarities to that identified by others with the common presence of P53, Ki67, CCNE, ERBB2 and CK5/6¹⁹ or CCNE, ERBB2, EGFR and CK5/6.²² Interestingly, this cluster also included CDH3/P-Cadherin, present in a “basal cluster” identified in gene expression analyses⁹ and previously shown to be overexpressed in a subgroup of breast carcinomas associated with higher proliferation rates and aggressive behavior.³⁵

Hierarchical clustering sorted tumors into three clusters that correlated with relevant histoclinical parameters, including histological type, SBR grade, ER status, ERBB2 status and the presence or absence of peritumoral vascular emboli. Correlations were found between the characteristics of these tumor clusters and their protein expression profiles. For example, the high number of grade III tumors in cluster B, as well as the high number of ERBB2-positive samples, agreed with the frequent strong expression of the “proliferation” cluster—which included ERBB2—and the “mitosis” cluster in these tumors. Conversely, 99% of cluster A1 samples were ER-positive, and showed a frequent strong expression of the “ER-related” cluster and low expression of the “proliferation cluster”.³²

Interestingly, the tumor clusters also correlated with a breast cancer classification recently proposed in two series of analyses that provided a new conceptual framework of mammary oncogenesis. First, phenotypic analyses have established a three-cell phenotypic classification of breast cancer cells.^{22, 36, 37} These authors suggested that biomarkers such as intermediate filaments cytokeratins (CK), encoded by a large number of keratin genes, are able to distinguish between distinct cell subpopulations within the mammary gland epithelial compartment. It has been proposed that “basal” cells contain mammary gland progenitor cells able to give raise to both “luminal” and “myoepithelial”³⁸ cells.(³⁹ for review) Progenitor cells express type II keratins CK5 and 6. In contrast, differentiated “luminal” cells express type II keratin CK8 and type I keratin CK18, which are also observed in normal simple and glandular epithelia. Luminal cells also express ER.^{10, 11} Use of tissue microarray screening has confirmed this emerging theory.^{19, 22} Second, recent gene expression analyses using DNA microarrays have led to a similar identification of subclasses of breast tumors that corresponded to the phenotypic classification.⁹⁻¹¹

These experiments concurred to establish a distinction between several types of epithelial cells in the mammary gland. The origin of the breast malignant cell remains unknown. Two major types of breast cancer may derive from basal/progenitor or luminal cells, respectively. Alternatively, most tumors may originate from pluripotent stem cells and reach different stages of differentiation.⁴⁰ Our results support this new classification model. Tumor cluster A1 may be approximated to a cluster of luminal cell-like tumors, with frequent strong expression of ER and CK8/18. Cluster B may consist of tumors with basal/progenitor, ER-negative characteristics, i.e. strong expression of CK5/6 and proliferation markers. A2 tumors, with an intermediate profile, may represent a transitory “baso-luminal” stage, or consist of tumors that have lost ER function. It can be expected that luminal A1 tumors, in which the bulk of cells are more differentiated and express ER-related cluster proteins, are of better prognosis, whereas more undifferentiated and proliferative basal B tumors are associated with poor prognosis. The significant differences in clinical outcome observed between the three defined tumor clusters in this study are consistent with this model and recent studies.^{9-11, 41} In addition, we discovered that lobular carcinomas are luminal-like tumors, and comprise differentiated luminal cells that express CK8/18.

Protein Expression Profiling Predicts Clinical Outcome of Breast Cancer

Thus, classical unsupervised hierarchical clustering applied to all tested proteins was able to identify biologically and clinically relevant classes of breast cancer. Recently, supervised methods have been successfully applied to gene expression data analysis in parallel with unsupervised approaches. In a second step, we thus developed a supervised method to identify the best combination within 26 proteins that would further improve the prognostic classification. To our knowledge, our study is the first application of such supervised methods to large-scale IHC data. We identified a 21-protein set which optimally classified patients into two classes (“good-prognosis” and “poor-prognosis class”) with significantly different long-term MFS.

Initially identified in a random learning set of 368 patients, this prognostic signature was validated in an independent set of 184 patients, showing its robustness. Our discriminator set included 10 proteins coded by genes identified across recent gene expression studies,⁷⁻¹⁵ as well as other proteins with unclear role in disease progression and sensitivity to systemic therapy. The prognostic value of the signature was increasingly accurate with the addition of other proteins as evidenced by univariate and multivariate analyses, further highlighting the strength of large-scale molecular analyses for understanding tumor heterogeneity through the identification of expression signatures.

The classification based on the 21-protein predictor was associated with a highly significant difference in clinical outcome. The 5-year MFS was 90% for patients of the “good-prognosis class” and only 62% for patients of the “poor-prognosis class.” When compared in multivariate analysis with classical prognostic factors and with each tested protein separately, our classification performed significantly better for predicting the occurrence of metastatic relapse. Such prognostic association persisted when applied to patients with lymph node-positive and lymph node-negative cancer.

Interestingly, the MFS of node-negative patients from the “poor-prognosis class” was similar to that of node-positive patients from the “good-prognosis class.” Notably, our molecular classification performed better than that defined by St-Gallen and NIH criteria for node-negative patients. This finding is of particular significance, since about 75% of node-negative patients candidate for adjuvant chemotherapy based on the St. Gallen/NIH criteria are currently thought to be over-treated.

Our 21-protein predictor assigned fewer node-negative patients to the “poor-prognosis class,” and their clinical outcome was more frequently unfavorable than it was for patients assigned to the high-risk class defined by St-Gallen or NIH criteria. Our predictor also performed well in patients irrespective of ER status. The 5-year MFS was 90% for ER-positive patients from the “good-prognosis class,” and 58% for ER-positive patients from the “poor-prognosis class,” suggesting our 21-protein set may provide more accurate clinical information than ER status alone, possibly reflecting functional differences in the ER pathway.

Additionally, our molecular classification conserved its predictive impact for patients independent of adjuvant systemic therapy. Since distant metastasis may be influenced by adjuvant therapy, we separately analyzed the 186 patients who did not receive any chemo- and hormone therapy, as well as the 133 patients who exclusively received adjuvant chemotherapy with anthracyclin-based regimen in most cases.

Interestingly, we found within the group of 186 untreated patients an odds ratio of 7.45 for metastatic relapse in the “poor-prognosis class” when compared with patients of the “good-prognosis class.” Similar discrimination was observed within the 133 patients treated with chemotherapy alone with a corresponding odds ratio of 3. Thus, the 21-protein signature may facilitate the selection of appropriate treatment options in early breast cancer patients. It may be an important clinical tool to circumvent unnecessary, toxic and costly treatment of node-negative patients, and it may help for selecting, among patients who need adjuvant chemotherapy, those who might benefit from standard protocol and those who would be candidates to other protocol or other form of systemic therapy.

Materials and Methods

Patients and Histological Samples

A consecutive series of 552 women with early (stage I, II or III) breast cancer treated at the Institut Paoli-Calmettes before December 1999 was studied using the TMA technology. The stage of disease was defined according to TNM classification (Union Internationale Contre le Cancer, UICC, TNM, 5^{th} edition). Patients with locally advanced, inflammatory or metastatic disease, or with previous history of cancer were not included. Tumors were invasive adenocarinomas including, according to the WHO histological typing, 388 ductal carcinomas (70%), 72 lobular (13%), 24 mixed (4%), 40 tubular (8%), 8 medullary (1%) and 20 other types (4%). Clinical annotation of each sample included patient age, axillary lymph node status, pathological tumor size, Scarff-Bloom-Richardson (SBR) grade, peritumoral vascular invasion, estrogen receptor (ER), progesterone receptor (PR) and ERBB2 status as evaluated by IHC with positivity cut-off values of 1% for hormone receptors and with 2 or 3+score (HercepTest kit scoring guidelines) for ERBB2. The characteristics of patients are listed in Table 2 (see first column only).

TABLE 2
Histoclinical characteristics of 552 breast cancer patients, according to the
membership to the “good-prognosis” or the “poor-prognosis class”
as defined using the expression of the 21-protein set.

| | All patients | | | |
|---|---|---|---|---|
| | (N = 552) | | | |
| | no. of patients | | | |
| | (% of | Good-prognosis | Poor-prognosis | |
| | evaluated | class* | class* | P- |
| Characteristics | cases) | (N = 358) | (N = 194) | value** |
| Age, years | | | | 0.87 |
| ≦50 | 153 (28) | 100 (28) | 53 (27) | |
| >50 | 399 (72) | 258 (72) | 141 (73) | |
| Lymph node metastasis | | | | 0.12 |
| 0 | 292 (53) | 199 (56) | 93 (49) | |
| 1-3 | 158 (29) | 103 (29) | 55 (29) | |
| >3 | 97 (18) | 55 (15) | 42 (22) | |
| Pathological tumor size | | | | 0.69 |
| pT1 | 245 (45) | 171 (48) | 74 (38) | |
| pT2 | 228 (42) | 136 (38) | 92 (48) | |
| pT3 | 75 (13) | 48 (14) | 27 (14) | |
| SBR grade | | | | <0.0001 |
| I | 181 (33) | 150 (42) | 31 (16) | |
| II | 229 (42) | 153 (43) | 76 (39) | |
| III | 139 (25) | 53 (15) | 86 (45) | |
| Peritumoral vascular | | | | 0.10 |
| invasion | | | | |
| absent | 345 (63) | 233 (65) | 112 (58) | |
| present | 206 (37) | 124 (35) | 82 (42) | |
| ER status | | | | <0.0001 |
| negative | 129 (23) | 12 (4) | 117 (60) | |
| positive | 422 (77) | 345 (96) | 77 (40) | |
| PR status | | | | <0.0001 |
| negative | 195 (35) | 67 (19) | 128 (66) | |
| positive | 355 (65) | 290 (81) | 65 (34) | |
| ERBB2 status | | | | <0.0001 |
| negative | 461 (87) | 317 (92) | 144 (77) | |
| positive | 70 (13) | 27 (8) | 43 (23) | |
| Chemotherapy | | | | 0.001 |
| no | 291 (53) | 208 (58) | 83 (43) | |
| yes | 261 (47) | 150 (42) | 111 (57) | |
| Hormone therapy | | | | <0.0001 |
| no | 286 (52) | 161 (47) | 125 (71) | |
| yes | 233 (48) | 181 (53) | 52 (29) | |
| Follow-up***, months | 57 (2, 182) | 56 (3, 181) | 58 (2, 182) | NS |
| median (range) | | | | |
| 5-year MFS | 80 [76.2-83.7] | 90 [86.0-93.3] | 62 [54.7-70.0] | <0.0001 |
| % [95% CI] | | | | |

*as defined using the 21-protein signature;
**P-values for the comparison of numbers of patients were calculated using the Chi-2 test, and P-values for the comparison of metastasis-free survival (MFS) were calculated using the log-rank test;
NS, not significant;
***calculated, for the 450 patients who did not experience metastatic relapse as a first event, from the date of diagnosis to the time of last follow-up;
CI denotes confidence interval.

Patients were treated according to the following guidelines: all had primary surgery that included complete resection of breast tumor (modified radical mastectomy in 28% of cases and lumpectomy in 72%) and axillary lymph node dissection; 96% of patients (including 100% of those treated with breast-conservative surgery) received adjuvant local-regional radiotherapy; 47% were given adjuvant chemotherapy (anthracyclin-based regimen in most cases), and 42% received adjuvant hormone treatment (tamoxifen for most cases). After completion of local-regional treatment, patients were evaluated at least twice per year for the first 5 years and at least annually thereafter. The median follow-up was 57 months (range, 2 to 182) after diagnosis for the 450 patients who did not experience metastatic relapse as a first event, 37 months (range, 4 to 151) for the 102 patients with metastasis as first event, and 51 months (range, 2 to 182) for all patients. The 5-year MFS rate was 80% [95% CI 76.2-83.7].

Tissue Microarrays Construction

TMA's were prepared as previously described²⁵ with slight modifications. For each tumor, three representative areas from the primary tumor were carefully selected from a hematoxylin-eosin stained section of a donor block. Core cylinders with a diameter of 0.6 mm each were punched from each of these areas and deposited into three separate recipient paraffin blocks using a specific arraying device (Beecher Instruments, Silver Spring, Md.). The technique of TMA allows the analysis of tumors and controls under identical experimental conditions. In addition to tumor tissues, the recipient block also received 10 normal breast tissue samples from 10 healthy women that underwent reductive mammary surgery and pellets from nine mammary cell lines. Five-μm sections of the resulting TMA block were made and used for IHC analysis after transfer onto glass slides. We previously assessed the reliability of the method by comparison with the standard immunohistochemical method for the usual prognostic parameters; the value of the kappa test was 0.95.²⁵

Selection of the 26 Markers

Selection of the proteins was performed according to the following criteria: known or potential importance in breast cancer and availability of a corresponding antibody that performed well in IHC on paraffin-embedded tissues. Twenty-six proteins were selected including hormone receptors (ER, PR), subclass markers (Cytokeratins), oncogenes and proliferation proteins (ERBB family members, BCL2, Cyclins, MIB1, FGFR1, Aurora A, Taxins), tumor suppressors (P53, FHIT), adhesion molecules (Cadherins, Catenins, Afadin), proteins from oncogenes of amplified genomic regions (ERBB2, CCND1, STK6), and other potential prognostic markers identified in specific studies or previous DNA microarray experiments (CCNE, GATA3, MUC1). Twelve out of the 26 proteins were mentioned as potential significant genes in RNA expression profiling studies in breast cancer.⁶⁻¹⁵ The characteristics of the antibodies used are listed in Table 3. When available, several antibodies were studied for comparison, and only the reagents that gave the best quality data were kept for the global analysis.

TABLE 3
Proteins tested by immunohistochemistry on TMAs and characteristics of the
corresponding antibodies.

| | Protein (acronym) | Antibody | Origin | Clone | Pretreatment | Dilution |
|---|---|---|---|---|---|---|
| 1 | Adhesion molecule | Mmab | Transduction | 35 | DTRS | 1/50 |
| | Afadin (AF6) | | laboratories | | (40 min, | |
| | | | | | 98° C.) | |
| 2 | Aurora A kinase | Mmab | C. Prigent, Rennes | / | DTRS | 1/25 |
| | (STK6/STK15) | | | | (40 min, | |
| | | | | | 98° C.) | |
| 3 | α-Catenin (CTNNA1) | Mmab | Zymed Laboratories | α CAT- | Citrate | 1/200 |
| | | | | 7A4 | buffer | |
| | | | | | (40 min, 98° C.) | |
| 4 | β-Catenin (CTNNB1) | Mmab | Transduction | 14 | Citrate | 1/2500 |
| | | | laboratories | | buffer | |
| | | | | | (40 min, 98° C.) | |
| 5 | Anti-apoptotic BCL2 | Mmab | Dako Corporation | 124 | Citrate | 1/100 |
| | | | | | buffer | |
| | | | | | (40 min, 98° C.) | |
| 6 | Cyclin D1 (CCND1) | Mmab | Zymed laboratories | AM29 | Citrate | 1/200 |
| | | | | | buffer | |
| | | | | | (40 min, 98° C.) | |
| 7 | Cyclin E (CCNE) | Mmab | Novocastra | 13A3 | Citrate | 1/50 |
| | | | Laboratories | | buffer | |
| | | | | | (40 min, 98° C.) | |
| 8 | Cytokeratins 5 and 6 | Mmab | Dako Corporation | D5/16B4 | DTRS | 1/10 |
| | (CK5/6) | | | | (40 min, | |
| | | | | | 98° C.) | |
| 9 | Cytokeratins 8 and 18 | Mmab | Zymed Laboratories | Zym5.2 | DTRS | 1/200 |
| | (CK8/18) | | | | (40 min, | |
| | | | | | 98° C.) | |
| 10 | Adhesion molecule E- | Mmab | Transduction | 36 | Citrate | 1/2000 |
| | Cadherin (CDH1) | | Laboratories | | buffer | |
| | | | | | (40 min, 98° C.) | |
| 11 | Epidermal growth | Mmab | Zymed Laboratories | 31G7 | Pepsin | 1/20 |
| | factor receptor (EGFR) | | | | (30 min, | |
| | | | | | 37° C.) | |
| 12 | Tyrosine kinase | Mmab | Novocastra | CB 11 | Citrate | 1/500 |
| | receptor ERBB2 | | Laboratories | | buffer | |
| | | | | | (40 min, 98° C.) | |
| 13 | Tyrosine kinase | Mmab | NeoMarkers | SGP1 | None | 1/40 |
| | receptor ERBB3 | | | | | |
| 14 | Tyrosine kinase | Mmab | NeoMarkers | HFR-1 | None | 1/50 |
| | receptor ERBB4 | | | | | |
| 15 | Estrogen receptor (ER) | Mmab | Novocastra | 6F11 | Citrate | 1/60 |
| | | | Laboratories | | buffer | |
| | | | | | (40 min, 98° C.) | |
| 16 | Fibroblast growth | Rpab | Santa Cruz | Sc-121 | DTRS | 1/200 |
| | factor receptor 1 | | Biotechnology | | (40 min, | |
| | (FGFR1) | | | | 98° C.) | |
| 17 | Fragile histidine | Rpab | Zymed Laboratories | ZR44 | Citrate | 1/300 |
| | triad (FHIT) | | | | buffer | |
| | | | | | (40 min, 98° C.) | |
| 18 | Transcription factor | Mmab | Santa Cruz | Sc-268 | Citrate | 1/100 |
| | GATA3 | | Biotechnology | | buffer | |
| | | | | | (40 min, 98° C.) | |
| 19 | MIB1/Ki67 | Mmab | Dako Corporation | Ki-67 | Citrate | 1/100 |
| | | | | | buffer | |
| | | | | | (40 min, 98° C.) | |
| 20 | Mucin 1 (MUC1) | Mmab | Transgene | H23 | None | 1/1000 |
| 21 | Tumor suppressor P53 | Mmab | Immunotech | DO-1 | Citrate | 1/4 |
| | | | | | buffer | |
| | | | | | (40 min, 98° C.) | |
| 22 | Adhesion molecule P- | Mmab | Transduction | 56 | DTRS | 1/75 |
| | Cadherin (CDH3) | | Laboratories | | (40 min, | |
| | | | | | 98° C.) | |
| 23 | Progesterone receptor | Mmab | Dako Corporation | PgR 636 | Citrate | 1/80 |
| | (PR) | | | | buffer | |
| | | | | | (40 min, 98° C.) | |
| 24 | Transforming acidic | Rpab | Upstate | 07-229 | DTRS | 1/200 |
| | coiled-coil 1/Taxin 1 | | Biotechnology | | (40 min, | |
| | (TACC1) | | | | 98° C.) | |
| 25 | Transforming acidic | Rpab | Upstate | 07-228 | DTRS | 1/40 |
| | coiled-coil 2/Taxin 2 | | Biotechnology | | (40 min, | |
| | (TACC2) | | | | 98° C.) | |
| 26 | Transforming acidic | Rpab | Upstate | 07-233 | DTRS | 1/100 |
| | coiled-coil 3/Taxin 3 | | Biotechnology | | (40 min, | |
| | (TACC3) | | | | 98° C.) | |

Mmab: mouse monoclonal antibody;
Rpab: rabbit polyclonal antibody;
DTRS: Dako target retrieval solution.

Immunohistochemical Analysis

IHC was carried out on five-μm sections of tissue fixed in alcohol formalin for 24 h and embedded in paraffin. Sections were deparaffinized in Histolemon (Carlo Erba Reagenti, Rodano, Italy) and rehydrated in graded alcohol. Antigen retrieval was accomplished by incubating the sections in pre-treatment solutions depending on the antibody used. Pretreatment conditions are listed in Table 3. The reactions were carried out using an autoimmunostainer (Dako Autostainer). Staining was performed at room temperature as follows: rehydrated tissues were washed in phosphate buffer, followed by quenching of endogenous peroxidase activity by treatment with 0.1% H₂O₂, slides, incubated with blocking serum (Dako) for 30 min., then with the affinity-purified antibody for one hour. After washing, slides were sequentially incubated with biotinylated antibody against rabbit IgG for 20 min. followed by streptadivin-conjugated peroxidase (Dako LSABR2 kit), then visualized with Diaminobenzidine (3-amino-9-ethylcarbazole). Slides were counter-stained with hematoxylin, coverslipped using Aquatex (Merck, Darmstadt, Germany) mounting solution, then evaluated under a light microscope by two pathologists. The results were expressed in terms of percentage (P) and intensity (I) of positive cells as previously described.²⁵ For each sample, the mean of the score of a minimum of two core biopsies was calculated. The results were then scored by the quick score (Q) (Q=P×I), except for ERBB2 status that was evaluated with the Dako scale (HercepTest™ kit scoring guidelines).

Quick score allowed separating tumors into two or three classes. Homogeneous classes were defined by grouping samples with an equivalent staining level according to the distribution curves as described.²⁵ Two classes (negative and positive) were defined for Afadin, α and β Catenins, BCL2, Cyclins D1 and E, Cytokeratins 5/6 and 8/18, EGFR, ERBB3, ERBB4, FGFR1, GATA3, MIB1, P53, P-Cadherin, PR and TACC3, with a positivity cut-off value of Q=1, except for Cyclin D1 and MIB1 with a positivity cut-off value of 10 and 20, respectively. Three classes were defined (negative, moderate and strong staining) for Aurora A, E-Cadherin, ER, FHIT, MUC1, TACC1, and TACC2, with negative (Q=0), moderate (0<Q≦100) or strong expression (100<Q≦300). For ERBB2, three classes (0/1+, 2+, 3+) were obtained with the Dako scale.

Data Analysis

A combination of exploratory unsupervised and supervised bioinformatic methods was used to analyze these immunohistochemical profiles. First, we applied unsupervised hierarchical clustering similar to that used in gene expression profiling studies. Data were reformatted using the following scoring system: −2 designated negative staining, 1 weakly positive staining, 2 strongly positive staining and missing data were left blank in the scored table. Hierarchical clustering investigates relationships between samples and between proteins, based on the similarity of sample immunoreactive scores. We used the Cluster program (average-linkage with Pearson correlation as similarity metric) and results were displayed with the TreeView software.²⁶

We then performed supervised analysis to identify the protein-set that best distinguished between two classes of samples with different clinical outcome. To simplify the analyses, the IHC scores were recorded as negative (negative staining) or positive (weakly and strong positive staining). The classifier was derived through training on a subset of chosen samples (⅔ of population, learning set) and then validated on the remaining subset (⅓ of population, validation set). The assignment of samples to each set was random, but the ratio between tumors with and without metastatic relapse was preserved. An exhaustive testing comprising all combinations of 1 to 5 proteins, as well as the complementary combinations of 21 to 25 proteins was performed to assess their ability to classify tumors into 2 classes (“poor-prognosis” and “good-prognosis”) in agreement with their clinical outcome.

Using the protein expression scores of each combination, we developed a “Metastasis Scoring” system that assigned to each tumor a probability to belong to the “poor-prognosis class” or the “good-prognosis class.” Consider a combination of N proteins P₁, K, P_{N} (where N ranges from 1 to 5 and 21 to 26) and two predefined classes X, Y of tumors within the learning set: X={X₁, K, X_{K}} includes samples with metastatic relapse during the follow-up and Y={Y₁, K, Y_{M}} includes samples without any metastatic relapse. For each protein combination tested, one tumor is represented as a ternary vector (e.g. X₁={X₁(P₁), K, X₁(P_{N})} where each component is scored 0 for missing data or +1/−1 for positive/negative IHC staining. Every tumor Z has a score S(Z) defined as follows. For each protein Pᵢ, we compute the frequencies of +1/−1 value in the X class (adjusted to avoid a 0 probability):

$${f}_{X}^{i} ( + 1 ) = \frac{card \{ k \text{:} {X}_{k} ( {P}_{i} ) = + 1 \} + 1}{card \{ k \text{:} {X}_{k} ( {P}_{i} ) \neq 0 \} + 2} and {f}_{X}^{i} ( - 1 ) = \frac{card \{ k \text{:} {X}_{k} ( {P}_{i} ) = - 1 \} + 1}{card \{ k \text{:} {X}_{k} ( {P}_{i} ) \neq 0 \} + 2}$$

where, for instance, card{k: Xₖ(Pᵢ)=+1} is the number of X tumors with positive IHC staining for protein Pᵢ. Similarly we compute the frequencies f_{Y}ⁱ(+1) and f_{Y}ⁱ(−1) in the Y class and we define f_{•}ⁱ(0)=1. The Metastasis Score of tumor Z is the log ratio of the joint probabilities:

$$S ( Z ) = {\sum }_{i = 1}^{N} log ( {f}_{X}^{i} ( Z ( {P}_{i} ) ) ) - {\sum }_{i = 1}^{N} log ( {f}_{Y}^{i} ( Z ( {P}_{i} ) ) ) .$$

Samples were then sorted according to their S(Z) score. The natural threshold that divides the population in 2 classes is S=0: if S(Z)>0 then Z is more similar to the class X and is predicted to belong to the “poor-prognosis class” and if S(Z)<0 then Z is more similar to the class Y and is predicted to belong to the “good-prognosis class.” The number of misclassifications (error rate) was defined as the number of X tumors classified in the “good-prognosis class” plus the number of Y tumors classified in the “poor-prognosis class.” The best classifier protein-set was that with the minimal rate of misclassified tumors.

Once identified, the prognostic power of the classifier was tested on the validation set by classifying the remaining independent tumors using the same approach. Finally, it was assessed on the whole population. For each tumor set, the prognostic impact was further estimated by univariate analyses that compared the rate of metastatic relapses within the two molecularly defined classes of tumors (Fisher exact test).

Statistical Methods

Distributions of molecular markers and other categorical variables were compared using either the standard Chi-2 test or Fisher exact test. The follow-up was calculated from the date of diagnosis to the time of metastasis as first event or time of last follow-up for censored patients. The end point was the metastasis-free survival (MFS), calculated from the date of diagnosis, first metastasis being scored as an event. All other patients were censored at the time of the last follow-up, death, recurrence of local or regional disease, or development of a second primary cancer, including contralateral breast cancer. Survival curves were derived from Kaplan-Meier estimates and Were compared by log-rank test. The influence of molecular grouping, adjusted for other factors including classical prognostic factors and significant IHC measurement, was assessed in multivariate analysis by the Cox proportional hazard models. Survival rates and odds ratios (OR) are presented with their 95% confidence intervals (95% CI). Statistical tests were two-sided at the 5% level of significance. All statistical tests were done using SAS Version 8.02.

Results

Expression Protein Profiling of Breast Cancers using Tissue Microarrays.

The expression of 26 proteins was studied by IHC on TMA containing 552 early stage breast tumor samples and controls (FIG. 4A). As expected, staining for all antibodies was homogeneous among the 10 normal breast samples (data not shown), but much more heterogeneous for tumor samples. Sixteen proteins were underexpressed in 12% (for MUC1) to 60% (for Aurora A) of cases, and overexpressed for 10 proteins in 11% (for Ki67/MIB1) to 66% (for ERBB4) of cases in cancerous tissues compared to normal samples. Examples of IHC staining are shown in FIG. 4 (panels B and C). Results are summarized in Table 4.

TABLE 4
Expression of proteins tested by immunohistochemistry in 552 early breast cancers
deposited on TMA and Kaplan-Meier analysis of the metastasis-free survival (MFS).

| | | Type of alteration in tumor | | | |
|---|---|---|---|---|---|
| | | samples*, frequency of | | | |
| | | alteration*, cell | No. of | | |
| Protein | | sublocalization | patients | 5-year MFS [95% CI] | P-value** |
| Afadin | negative | Downregulated, 14%, membrane | 48 | | 0.13 |
| | positive | and cytoplasm | 300 | | |
| Aurora A | negative | Downregulated, 60%, nucleus | 267 | | 0.25 |
| | positive | | 177 | | |
| α-Catenin | negative | Downregulated, 30%, membrane | 105 | 66.9 [56.8-77.0] | 0.0046 |
| | positive | | 267 | 84.9 [80.1-89.7] | |
| β-Catenin | negative | Downregulated, 40%, membrane | 152 | 72.2 [64.2-80.1] | 0.031 |
| | positive | | 229 | 82.1 [76.9-88.8] | |
| BCL2 | negative | Downregulated, 21%, | 88 | 57.6 [45.3-69.9] | <0.0001 |
| | positive | cytoplasm | 324 | 83.9 [79.4-88.4] | |
| Cyclin D1 | ≦10 | Upregulated, 21%, nucleus | 380 | | 0.82 |
| | >10 | | 101 | | |
| Cyclin E | negative | Upregulated, 15%, nucleus | 363 | | 0.44 |
| | positive | | 66 | | |
| Cytokeratin | negative | Upregulated, 32%, membrane | 246 | | 0.06 |
| 5/6 | positive | and cytoplasm | 125 | | |
| Cytokeratin | negative | Downregulated, 14%, membrane | 29 | | 0.07 |
| 8/18 | positive | and cytoplasm | 456 | | |
| E-Cadherin | negative | Downregulated, 17%, membrane | 61 | | 0.41 |
| | positive | | 424 | | |
| EGFR | negative | Upregulated, 21%, membrane | 349 | | 0.45 |
| | positive | | 92 | | |
| ERBB2 | 0-1 | Upregulated, 12%, membrane | 433 | 81.9 [77.8-86.0] | 0.030 |
| | 2-3 | | 60 | 64.2 [48.8-79.6] | |
| ERBB3 | negative | Upregulated, 58%, cytoplasm | 158 | | 0.29 |
| | positive | and membrane | 223 | | |
| ERBB4 | negative | Upregulated, 66%, cytoplasm | 135 | | 0.99 |
| | positive | and membrane | 260 | | |
| Estrogen | negative | Downregulated, 24%, nucleus | 133 | 67.0 [58.1-75.9] | <0.0001 |
| receptor | positive | | 408 | 85.2 [81.3-89.1] | |
| FGFR1 | negative | Upregulated, 45%, cytoplasm | 193 | | 0.92 |
| | positive | and membrane | 233 | | |
| FHIT | negative | Downregulated, 16%, | 69 | | 0.37 |
| | positive | cytoplasm | 353 | | |
| GATA3 | negative | Downregulated, 45%, nucleus | 170 | 69.7 [61.9-77.5] | 0.0006 |
| | positive | | 268 | 85.1 [80.3-89.9] | |
| MIB1/Ki67 | ≦20 | Upregulated, 11%, nucleus | 406 | 83.4 [79.2-87.5] | <0.0001 |
| | >20 | | 53 | 56.0 [39.4-72.5] | |
| Mucin 1 | negative | Downregulated, 12%, | 53 | | 0.22 |
| | positive | cytoplasm and membrane | 390 | | |
| P53 | negative | Upregulated, 26%, nucleus | 383 | 82.2 [77.8-86.5] | 0.003 |
| | positive | | 132 | 71.2 [62.5-80.0] | |
| P-Cadherin | negative | Downregulated, 55%, | 248 | | 0.28 |
| | positive | membrane | 207 | | |
| Progesterone | negative | Downregulated, 36%, nucleus | 185 | 71.7 [64.4-79.0] | 0.0007 |
| receptor | positive | | 333 | 84.9 [80.5-89.3] | |
| TACC1 | negative | Downregulated, 47%, | 208 | | 0.88 |
| | positive | cytoplasm | 231 | | |
| TACC2 | negative | Downregulated, 27%, | 107 | 72.8 [63.7-81.9] | 0.048 |
| | positive | cytoplasm | 288 | 80.3 [74.8-85.7] | |
| TACC3 | negative | Downregulated, 39%, | 184 | | 0.20 |
| | positive | cytoplasm | 286 | | |

*as compared to 10 normal breast samples.
**P-values for the comparison of MFS were calculated using the log-rank test.
CI denotes confidence interval.

Unsupervised Hierarchical Classification of 552 Breast Tumors Upon Protein Expression Profiling
Hierarchical Clustering

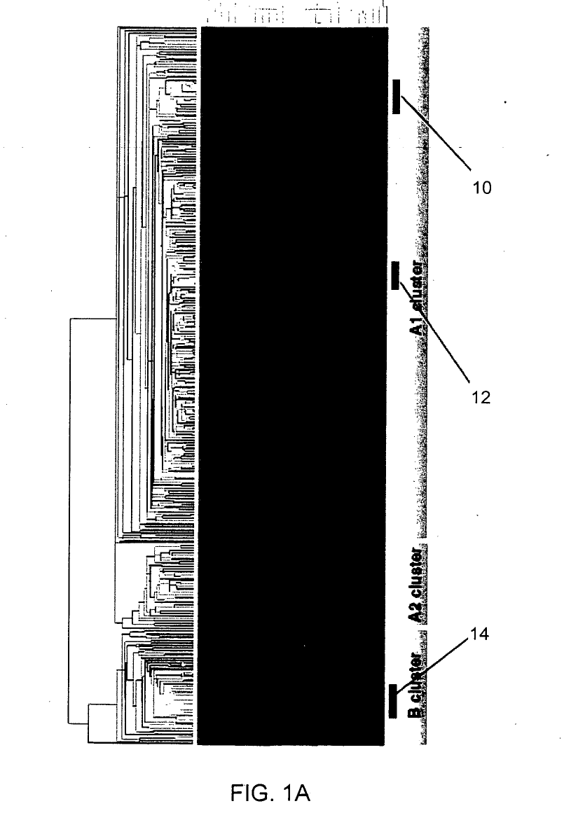

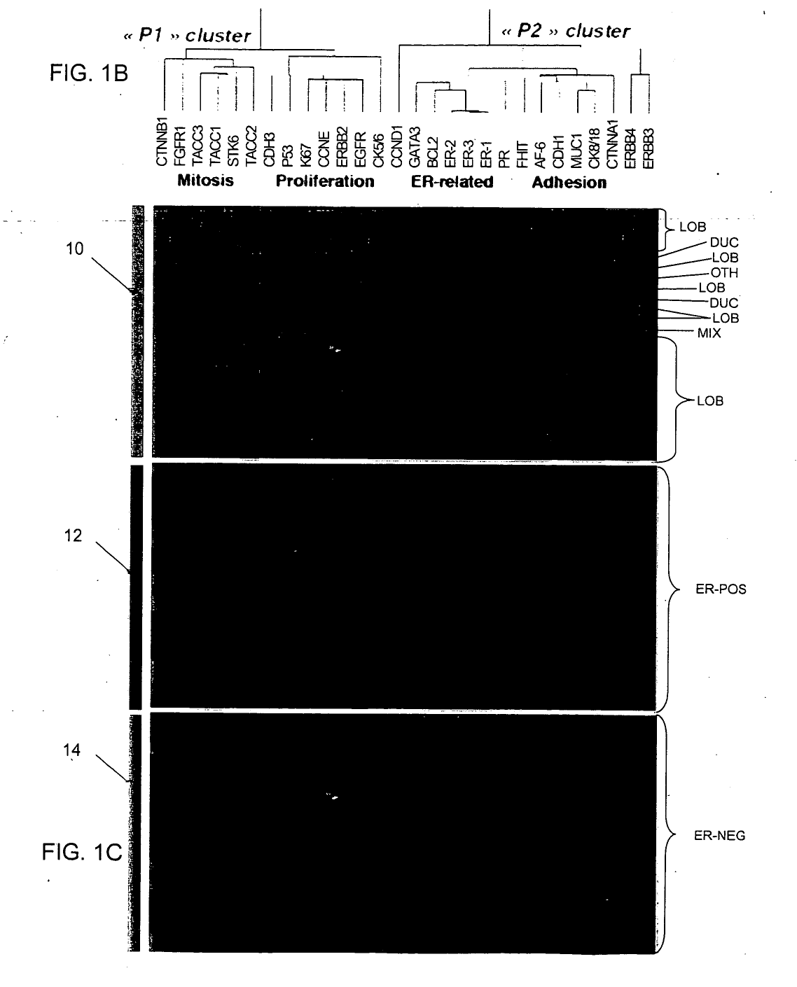

The overall expression patterns for the 552 samples were first analyzed with hierarchical clustering. Results are displayed in a color-coded matrix in FIG. 1A. The clustering algorithm orders proteins on the horizontal axis and samples on the vertical axis on the basis of similarity of their expression profiles. This similarity is shown as a dendrogram where the length of branch between two elements reflects their degree of relatedness. Protein expression scores are represented according to a color scale: red for strong positive staining, brown for weak positive staining and green for negative staining. Despite significantly heterogeneous expression, such combinatorial analysis and color display highlighted groups of correlated proteins across correlated samples.

FIG. 1B displays the dendrogram of related proteins. As expected, the three interpretations of ER staining made independently by two pathologists were highly correlated (R² between 0.87 and 0.96) (FIG. 1C, middle and bottom panels). Furthermore, there was a high degree of concordance for expression of ER between IHC on full sections and on TMA (p<0.0001, Chi-2 test). Two major protein clusters—designated “P1” and “P2”—were identified (FIG. 1B). These clusters were further divided into smaller sub-groups including a cluster (thereafter designated “ER-related cluster”) of ER-associated proteins (PR, BCL2, GATA3) and an “adhesion cluster” (E-Cadherin, α-Catenin, Afadin). We²⁷ have demonstrated that Aurora A (STK6) and Taxins (TACC1-3) are interacting partners and involved in cell division. This translated in the formation of a third cluster (thereafter designated “mitosis cluster”). The fourth cluster (thereafter designated “proliferation cluster”) defined by the routinely used marker Ki67/MIB1, revealed that proteins such as EGFR, ERBB2, P53 and the G1 cyclin CCNE are preferentially overexpressed in tumors undergoing rapid growth.

The combined protein expression patterns defined two major clusters of tumors designated cluster A (462 cases) and cluster B (89 cases) in FIG. 1 (1 case that clustered outside of the 2 clusters was excluded from further analysis). Cluster A could be further subdivided into two subclusters, A1 (393 cases) and A2 (89 cases). Globally, cluster A1 tumors displayed a strong expression of the “ER cluster” and the “adhesion cluster” and a low expression of the “proliferation cluster” in most of cases, whereas the “mitosis cluster” was strongly expressed in about 50% of samples. In general, cluster B tumors displayed overall a low expression of the “ER cluster” but a strong expression of the three other protein clusters. Cluster A2 included ER-positive and ER-negative tumors that displayed an intermediate profile characterized overall by strong expression of the “adhesion cluster” and a low expression of the “ER cluster,” the “proliferation cluster” and the “mitosis cluster.”

Correlation with Histoclinical Parameters and Survival

We identified correlations between tumor clusters and relevant biopathological parameters. In each cluster, the most frequent histological type was the ductal type. However, in cluster A1, 19% of samples were of the lobular type compared with 12% in cluster A2 and only 7% in cluster B (p=0.03; Chi-2 test). FIG. 1C (top panel) shows, within cluster A1, a subcluster of 24 tumors that includes 21 lobular or mixed (lobular/ductal) carcinomas with low expression of E-Cadherin, consistent with a previous report.²⁹ Correlation also existed with SBR grade; in cluster A1, 41% of cases were grade 1 and 15% were grade III compared with 23% and 35% in cluster A2, and 7% and 63% in cluster B (p<0.0001; Chi-2 test), respectively. In cluster B, samples were more likely to be ERBB2-positive (2+ or 3+ in IHC, 36% of cases) compared with 8% in cluster A1 and 12% in cluster A2 (p<0.0001, Chi-2 test). Conversely, cluster A1 samples were more likely to be ER-positive (99% of cases) compared with 35% in cluster A2 and 10% in cluster B (p<0.0001, Chi-2 test). Finally, peritumoral vascular emboli were more frequent in A2 tumors (53% of cases) than in B (37%) and A1 (35%) tumors (p=0.02, Chi-2 test). Interestingly, no correlation was found with age of patients, pathological size of tumors, and axillary lymph node status.

Importantly, the tumor clusters correlated with clinical outcome. With a median follow-up of 57 months, the 5-year MFS was significantly different (p<0.0001, log-rank test) between cluster A1 (54 metastases, 86% MFS [95% CI 82.1-89.9]), cluster A2 (21 metastases, 68% MFS [95% CI 79.9-56.5]) and cluster B (26 metastases, 66% MFS [95% CI 54.3-77.6]) (data not shown).

Supervised Analysis and Clinical Outcome

We developed a supervised analysis method to search for smaller sets of discriminator proteins that might improve our prognostic classification. Analysis was conducted using two equivalent but independent tumor sets (learning and validation sets).

Supervised Analysis and Classification of Patients

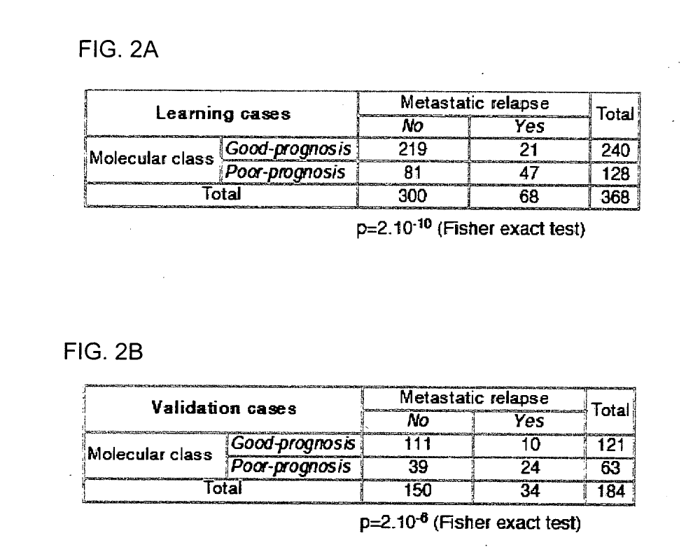

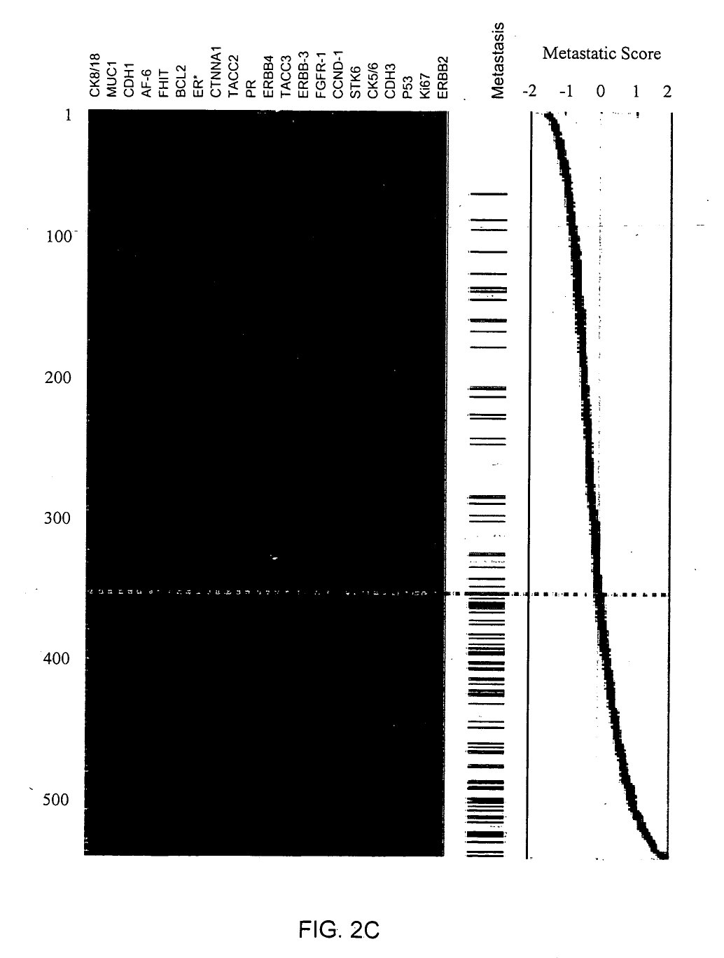

The learning set of samples (n=368) allowed the identification of a combination of proteins (protein expression signature) that correlated with long-term MFS. The number of proteins in the “metastatic predictor” was optimized by iteratively testing all combinations of 1 to 5 proteins and the complementary combinations of 21 to 25 proteins and by assessing their ability for correct classification of samples using a “Metastatic Score.” The optimal combination for these tumors contained 21 proteins (FIG. 2C). Examples of IHC staining for these 21 proteins are shown in FIG. 4B. Samples from the learning set were ordered using the “Metastatic Score.” Two classes of samples (“poor-prognosis class,” positive scores and “good-prognosis class,” negative scores) were defined using a cut-off value of 0. As shown in FIG. 2A, the classifier predicted rather successfully the actual clinical outcome of patients: 47 out of the 128 patients (37%) with positive score displayed metastatic relapse whereas only 21 out of the 240 (9%) with negative score experienced metastasis during follow-up (odds ratio, OR=6.1 [95% CI 3.3-11.3], p<0.0001, Fisher exact test).

We then shown the ability of this multiprotein signature to predict prognosis in an independent set of 184 patients (validation set). Using the same threshold for the “Metastatic Score” previously described, we identified two classes of patients that strongly correlated with clinical outcome. There were 24 metastatic relapses out of the 63 patients (38%) in the “poor-prognosis class” and only 10 out of the 121 (8%) in the “good-prognosis class” (odds ratio, OR=6.8 [95% CI 2.8-17.3], p<0.0001, Fisher exact test) (FIG. 2B). These results confirmed and validated the predictive capacity and robustness of our 21-protein signature.

When all 552 cases (learning and validation cases) were analyzed together, the predictor correlated well with long-term MFS. FIG. 2C shows the expression profiles of the 21 proteins in the 552 tumors in a color-coded matrix. Samples are ordered from top to bottom according to their increasing “Metastatic Score” and proteins from left to right according to decreasing ΔP (ΔP is the difference between the probability of positive staining and the probability of negative staining in non-metastatic samples). The orange dashed line indicates the threshold 0 that separates the two classes, “good-prognosis” (above the line) and “poor-prognosis” (under the line).

Correlation of Molecular Classification with Histoclinical Parameters and Survival

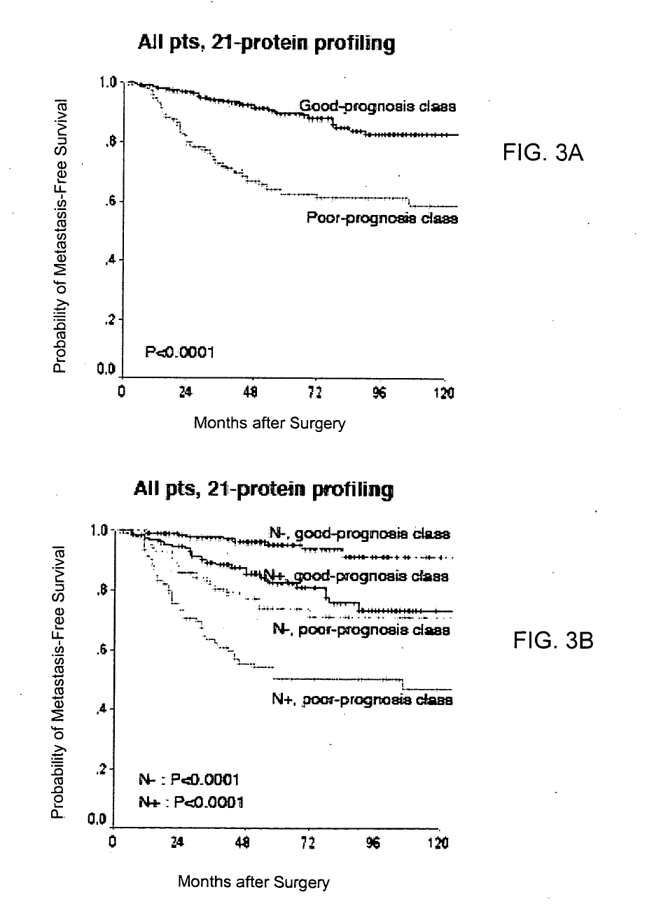

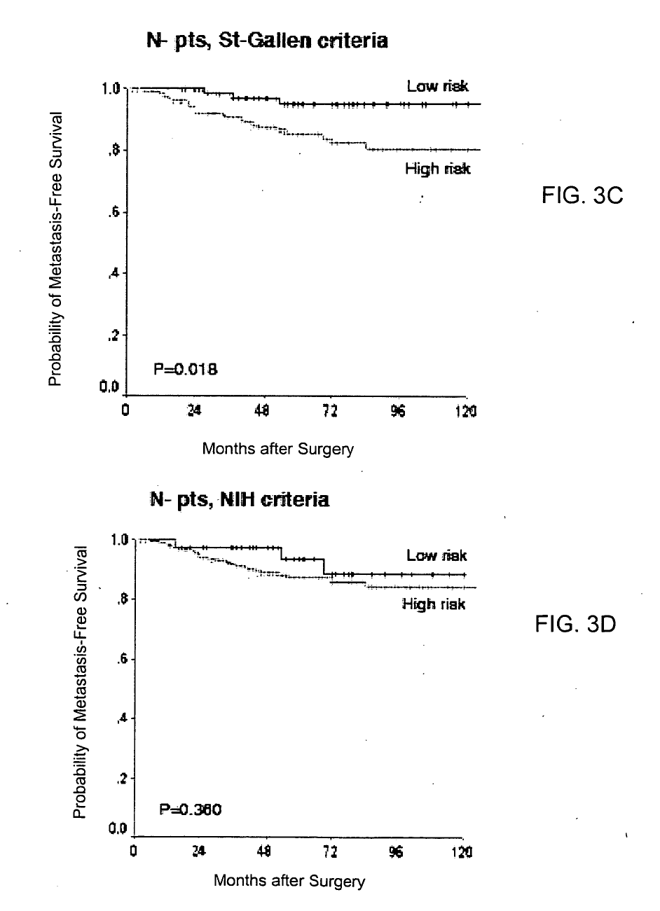

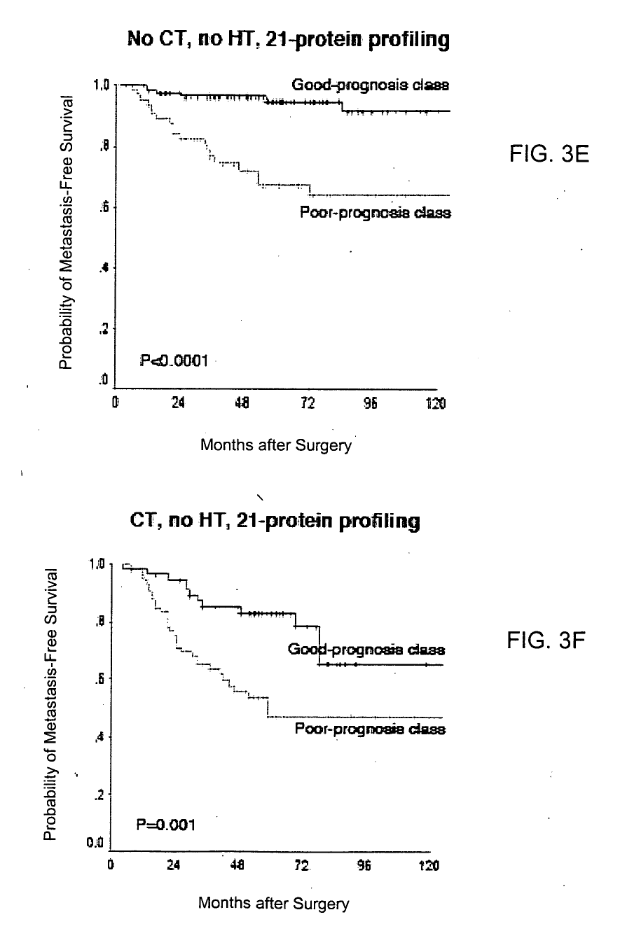

Table 2 (see the three last columns) shows the characteristics of patients in each class. The histoclinical parameters significantly associated with this classification were SBR grade (p<0.0001, Chi-2 test), hormone receptor status (p<0.0001, Fisher exact test), ERBB2 status (p<0.0001, Fisher exact test), and whether patients received adjuvant chemotherapy (p=0.001, Fisher exact test) or hormone therapy (p<0.0001, Fisher exact test). There was no correlation with patient age, tumor size, and number of involved lymph nodes. In contrast, a strong correlation with clinical outcome was observed (FIG. 2C): 65 of 194 patients (34%) assigned to the “poor-prognosis class” displayed metastatic relapse whereas only 37 of 358 (10%) assigned to the “good-prognosis class” experienced metastasis during follow-up (odds ratio, OR=4.4 [95% CI 2.7-7.0], p<0.0001, Fisher exact test). The 5-year MFS was 62% [95% CI 54.7-70.0] in the “poor-prognosis class,” and 90% [95% CI 86.0-93.3] in the “good-prognosis class” (p<0.0001, log-rank test) (FIG. 3A).

Survival and Lymph Node Status

Our protein expression signature also classified the 255 patients with node-positive disease into two classes that correlated with clinical outcome. In the “good-prognosis class,” 28 out of 158 patients experienced metastatic relapse during follow-up as compared with 43 out of 97 in the “poor-prognosis class” (odds ratio, OR=3.7 [95% CI 2.0-6.8], p<0.0001, Fisher exact test) (FIG. 3B).

The same was true for the 292 patients with node-negative breast cancer. In this group, the odds ratio for metastasis was 6.5 ([95% CI 2.7-16.8], p<0.0001, Fisher exact test) among the 93 women from the “poor-prognosis class,” as compared with the 199 women from the “good-prognosis class” (FIG. 3B). As shown, there was no significant difference for MFS between the 158 node-positive patients from the “good-prognosis class” and the 93 node-negative patients from the “poor-prognosis class” (p=0.142, log-rank test).

We compared our prognostic classification of node-negative patients with those provided by the consensus criteria established during the St-Gallen and NIH conferences.^{3, 4} These criteria classified all 292 patients into two groups (low risk versus high risk) (FIGS. 3C and 3D). Our multiprotein signature classified many more patients into the “good-prognosis class” (199 vs 80 vs 43, respectively) and less patients in the “poor-prognosis class” (93 vs 209 vs 245) as compared with St-Gallen and NIH classifications, and interestingly, with a percentage of metastatic relapse similar in the classes with low risk (4.5% vs 5% vs 7%, respectively), but greater in the classes with high risk (24% vs 13% vs 11%, respectively). In fact, the low-risk group and the high-risk group defined according to consensual criteria could further be subdivided in prognostic subgroups when the 21-protein signature was applied (data not shown).

Survival and Estrogen Receptor Status.

The same analysis was separately applied to ER-positive and ER-negative tumors. In the ER-positive group (n=422), 35 of 345 patients from the “good-prognosis class” displayed metastatic relapse as compared with 29 of 77 from the “poor-prognosis class” (odds ratio, OR=5.4 [95% CI 2.8-9.9], p=<0.0001, Fisher exact test). The corresponding 5-year MFS were 90% [95% CI 85.9-93.3] and 58% [95% CI 45.4-70.6], respectively (p<0.0001, log-rank test) (data not shown). The same trend was observed, although not significant (p=0.21, log-rank test), for the 129 ER-negative tumors with 5-year MFS of 91% [95% CI 76.0-100.0] and 66% [95% CI 56.0-75.1], respectively.

Survival and Adjuvant Systemic Therapy

Since the occurrence of metastatic relapse may be influenced by the delivery of adjuvant systemic therapy, the classification based on our 21-protein signature was applied to 186 women who received neither chemotherapy nor hormone therapy after local-regional treatment. Importantly, the 21-protein signature successfully predicted prognosis in these patients: 6 metastatic relapses of 119 patients in the “good-prognosis class” and 19 of 67 in the “poor-prognosis class” (odds ratio, OR=7.4 [95% CI 2.6-23.9], p<0.0001, Fisher exact test) (FIG. 3E).

Similar results were observed when we focused on the 133 patients who received adjuvant chemotherapy without hormone therapy. In the “good-prognosis class,” 12 of the 58 patients displayed metastatic relapse whereas 33 of 75 experienced metastasis in the “poor-prognosis class” (odds ratio, OR=3 [95% CI 1.3-7.2], p=0.006 Fisher exact test) (FIG. 3F).

Uni- and Multivariate Prognostic Analysis

We finally compared the prognostic ability of our molecular grouping of tumors with classical histoclinical factors and individual protein markers. In univariate analysis, the histoclinical factors that correlated with MFS (p<0.05, log-lank test) were pathological tumor size (≦20 mm, >20), tumor grade (SBR I, II, III), number of positive axillary lymph nodes (0, 1-3, ≧4), and peritumoral vascular invasion (negative, positive). Proteins significantly correlated to MFS were BCL2 (p<0.0001), GATA3 (p=0.0006), MIB1 (p<0.0001), ER (p<0.0001), PR (p=0.0007), P53 (p=0.003) and α-Catenin (p=0.005) (Table 5).

TABLE 5
Cox proportional-hazards multivariate analyses in
metastasis-free survival (n = 552).

| Variable | Hazard ratio [95% CI] | P-value |
|---|---|---|
| Molecular classification (21-protein set) | | |
| “good-prognosis class” | 1 | <0.0001 |
| “poor-prognosis class” | 2.20 [1.25-3.89] | |
| Tumor size | | |
| ≦20 mm | 1 | |
| >20 mm | 3.17 [1.74-5.75] | 0.0003 |
| Axillary lymph node metastasis | | |
| ≦3 | 1 | 0.0018 |
| >3 | 2.48 [1.45-4.25] | |
| MIB1/Ki67 status | | |
| negative | 1 | |
| positive | 2.38 [1.30-4.33] | 0.0030 |
| Hormone therapy | | |
| no | 1 | |
| yes | 0.48 [0.27-0.87] | 0.0137 |

CI denotes confidence interval.

The influence on the risk of distant metastasis of our multiprotein-based grouping, adjusted for other prognostic factors, was assessed in multivariate analysis by the Cox proportional hazards model. The parameters entered in the model were dichotomised and included the classification based on the discriminator 21-protein set (“good-prognosis class” and “poor-prognosis class”), age of patients (≦50 years, >50 years), number of positive axillary lymph nodes (0, 1-3, ≧4), pathological tumor size (≦20 mm, >20), tumor grade (SBR I, II, III), estrogen receptor status (negative, positive), progesterone receptor status (negative, positive), peritumoral vascular invasion (negative, positive), chemotherapy (delivery or not), hormone therapy (delivery or not) and each of the proteins (negative, positive) significantly associated with survival in univariate analyses. Results are shown in Table 5. Several independent factors predictive of distant metastasis as first event were evidenced including the prognosis signature based on the 21-protein combination, pathological size of tumors, axillary lymph node status (only when dichotomized ≦3 vs >3), Ki67/MIB1 status and delivery of hormone therapy. However, the 21-protein signature was the strongest predictor with a hazard ratio of 2.2 for “poor-prognosis class” patients, compared to “good-prognosis class” patients ([95% CI 1.25-3.89], p<0.0001).

### REFERENCES

The references below and the subject matter therein is incorporated herein by reference.
1. Tamoxifen for early breast cancer: an overview of the randomised trials. Early Breast Cancer Trialists' Collaborative Group. Lancet 1998; 351:1451-67.
2. Polychemotherapy for early breast cancer: an overview of the randomised trials. Early Breast Cancer Trialists' Collaborative Group. Lancet 1998; 352:930-42.
3. Eifel P, Axelson J A, Costa J, et al. National Institutes of Health Consensus Development Conference Statement: adjuvant therapy for breast cancer, Nov. 1-3, 2000. J Natl Cancer Inst 2001; 93:979-89.
4. Goldhirsch A, Glick J H, Gelber R D, Coates A S, Senn H J. Meeting highlights: International Consensus Panel on the Treatment of Primary Breast Cancer. Seventh International Conference on Adjuvant Therapy of Primary Breast Cancer. J Clin Oncol 2001; 19:3817-27.
5. Leyland-Jones B. Trastuzumab: hopes and realities. Lancet Oncol 2002; 3:137-44.
6. Bertucci F, Viens P, Hingamp P, Nasser V, Houlgatte R, Birnbaum D. Breast cancer revisited using DNA array-based gene expression profiling. Int J Cancer 2003; 103:565-71.
7. Bertucci F, Houlgatte R, Benziane A, et al. Gene expression profiling of primary breast carcinomas using arrays of candidate genes. Hum Mol Genet 2000; 9:2981-2991.
8. Bertucci F, Nasser V, Granjeaud S, et al. Gene expression profiles of poor-prognosis primary breast cancer correlate with survival. Hum Mol Genet 2002; 11:863-72.
9. Perou C M, Sorlie T, Eisen M B, et al. Molecular portraits of human breast tumours. Nature 2000; 406:747-52.
10. Sorlie T, Tibshirani R, Parker J, et al. Repeated observation of breast tumor subtypes in independent gene expression data sets. Proc Natl Acad Sci USA 2003; 100:8418-23.
11. Sotiriou C, Neo S Y, McShane L M, et al. Breast cancer classification and prognosis based on gene expression profiles from a population-based study. Proc Natl Acad Sci USA 2003; 100:10393-8.
12. van de Vijver M J, He Y D, van't Veer L J, et al. A gene-expression signature as a predictor of survival in breast cancer. N Engl J Med 2002; 347:1999-2009.
13. van't Veer L J, Dai H, van De Vijver M J, et al. Gene expression profiling predicts clinical outcome of breast cancer. Nature 2002; 415:530-6.
14. Huang E, Cheng S H, Dressman H, et al. Gene expression predictors of breast cancer outcomes. Lancet 2003; 361:1590-6.
15. Cheng Q, Lau W M, Tay S K, Chew S H, Ho T H, Hui K M. Identification and characterization of genes involved in the carcinogenesis of human squamous cell cervical carcinoma. Int J Cancer 2002; 98:419-26.
16. Hoos A, Cordon-Cardo C. Tissue microarray profiling of cancer specimens and cell lines: opportunities and limitations. Lab Invest 2001; 81:1331-8.
17. Kononen J, Bubendorf L, Kallioniemi A, et al. Tissue microarrays for high-throughput molecular profiling of tumor specimens. Nat Med 1998; 4:844-7.
18. Richter J, Wagner U, Kononen J, et al. High-throughput tissue microarray analysis of cyclin E gene amplification and overexpression in urinary bladder cancer. Am J Pathol 2000; 157:787-94.
19. Callagy G, Cattaneo E, Daigo Y, et al. Molecular classification of breast carcinomas using tissue microarrays. Diagn Mol Pathol 2003; 12:27-34.
20. Hsu F D, Nielsen T O, Alkushi A, et al. Tissue microarrays are an effective quality assurance tool for diagnostic immunohistochemistry. Mod Pathol 2002; 15:1374-80.

21. Liu C L, Prapong W, Natkunam Y, et al. Software tools for high-throughput analysis and archiving of immunohistochemistry staining data obtained with tissue microarrays. Am J Pathol 2002; 161:1557-65.
22. Korsching E, Packeisen J, Agelopoulos K, et al. Cytogenetic alterations and cytokeratin expression patterns in breast cancer: integrating a new model of breast differentiation into cytogenetic pathways of breast carcinogenesis. Lab Invest 2002; 82:1525-33.
23. Alkushi A, Irving J, Hsu F, et al. Immunoprofile of cervical and endometrial adenocarcinomas using a tissue microarray. Virchows Arch 2003; 442:271-7.
24. Nielsen T O, Hsu F D, O'Connell J X, et al. Tissue Microarray Validation of Epidermal Growth Factor Receptor and SALL2 in Synovial Sarcoma with Comparison to Tumors of Similar Histology. Am J Pathol 2003; 163:1449-56.
25. Ginestier C, Charaffe-Jauffret E, Bertucci F, et al. Interest and limitations of tissue-microarrays for validation of breast tumor markers selected upon cDNA array analysis. Am J Pathol 2002; 161:1223-1233.
26. Eisen M B, Spellman P T, Brown P O, Botstein D. Cluster analysis and display of genome-wide expression patterns. Proc Natl Acad Sci USA 1998; 95:14863-8.
27. Conte N, Delaval B, Ginestier C, et al. The TACC1-chTOG-Aurora A protein complex in breast cancer. Oncogene in press.
28. Giet R, McLean D, Descamps S, et al. Drosophila Aurora A kinase is required to localize D-TACC to centrosomes and to regulate astral microtubules. J Cell Biol 2002; 156:437-51.
29. Droufakou S, Deshmane V, Roylance R, Hanby A, Tomlinson I, Hart I R. Multiple ways of silencing E-cadherin gene expression in lobular carcinoma of the breast. Int J Cancer 2001; 92:404-8.
30. Teixeira C, Reed J C, Pratt M A. Estrogen promotes chemotherapeutic drug resistance by a mechanism involving Bcl-2 proto-oncogene expression in human breast cancer cells. Cancer Res 1995; 55:3902-7.
31. Menard S, Fortis S, Castiglioni F, Agresti R, Balsari A. HER2 as a prognostic factor in breast cancer. Oncology 2001; 61:67-72.
32. Fisher E R, Osborne C K, McGuire W L, et al. Correlation of primary breast cancer histopathology and estrogen receptor content. Breast Cancer Res Treat 1981; 1:37-41.
33. Ginestier C, Bardou V J, Popovici C, et al. Loss of FHIT protein expression is a marker of adverse evolution in good prognosis localized breast cancer. Int J Cancer 2003; 107:854-62.
34. Hui R, Cornish A L, McClelland R A, et al. Cyclin D1 and estrogen receptor messenger RNA levels are positively correlated in primary breast cancer. Clin Cancer Res 1996; 2:923-8.
35. Paredes J, Milanezi F, Reis-Filho J S, Leitao D, Athanazio D, Schmitt F. Aberrant P-cadherin expression: is it associated with estrogen-independent growth in breast cancer? Pathol Res Pract 2002; 198:795-801.
36. Lakhani S R, Chaggar R, Davies S, et al. Genetic alterations in ‘normal’ luminal and myoepithelial cells of the breast. J Pathol 1999; 189:496-503.
37. Dontu G, Al-Hajj M, Abdallah W M, Clarke M F, Wicha M S. Stem cells in normal breast development and breast cancer. Cell Prolif 2003; 36 Suppl 1:59-72.
38. Lakhani S R, O'Hare M J. The mammary myoepithelial cell—Cinderella or ugly sister? Breast Cancer Res 2001; 3:1-4.
39. Boecker W, Buerger H. Evidence of progenitor cells of glandular and myoepithelial cell lineages in the human adult female breast epithelium: a new progenitor (adult stem) cell concept. Cell Prolif 2003; 36 Suppl 1:73-84.

40. Al-Hajj M, Wicha M S, Benito-Hernandez A, Morrison S J, Clarke M F. Prospective identification of tumorigenic breast cancer cells. Proc Natl Acad Sci USA 2003; 100:3983-8.
41. van de Rijn M, Perou C M, Tibshirani R, et al. Expression of cytokeratins 17 and 5 identifies a group of breast carcinomas with poor clinical outcome. Am J Pathol 2002; 161:1991-6.
42. Brazma A, Vilo J. Gene expression data analysis. FEBS Lett 2000; 480:17-24.

1) A method for analyzing differential protein expression associated with histopathologic features of breast disease comprising detecting overexpression or underexpression of a pool of proteins in breast tissues or cells, the pool comprising at least one of a protein set comprising:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, estrogen receptor, FGFR1, FHIT, GATA3, Ki67, Mucin 1, P53, P-Cadherin, Progesterone receptor, TACC1, TACC2, TACC3, Cytokeratin 6, Cytokeratin 18, Ang1, AuroraB, BCRP1, CathepsinD, CD10, CD44, CK14, Cox2, FGF2, GATA4, Hif1a, MMP9, MTA1, NM23, NRG1a, NRG1beta, P27, Parkin, PLAU, S100, SCRIBBLE, Smooth Muscle Actin, THBS1, TIMP1, VEGFc and Vimentine.

2) A method for analyzing differential protein expression associated with histopathologic features of breast disease comprising detecting overexpression or underexpression of a pool of proteins in breast tissues or cells, the pool comprising at least one of a protein set comprising:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2 and TACC3.

3) A method for analyzing differential protein expression associated with histopathologic features of breast disease comprising detecting overexpression or underexpression of a pool of protein in breast tissues comprising at least one of a protein set comprising:
Afadin, Aurora A, a-Catenin, BCL2, Cyclin D1, Cytokeratin 5/6, Cytokeratin 8/18, E-Cadherin, ERBB2, ERBB3, ERBB4, Estrogen receptor, FGFR1, FHIT, Ki67, Mucin 1, P53, P-Cadherin, Progesterone receptor, TACC2 and TACC3.

4) The method according to claims 1 to 3, wherein the protein set comprises:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2 and TACC3.

5) The method according to claims 1 to 3, comprising detecting overexpression of at least one of the proteins:
EGFR, P53, Ki67, FGFR1, ERBB2, ERBB3, ERBB4, Cyclin D1, Cyclin E and Cytokeratin 5/6.

6) The method according to claim 4, comprising detecting overexpression of at least one of the proteins:
EGFR, P53, Ki67, FGFR1, ERBB2, ERBB3, ERBB4, Cyclin D1, Cyclin E and Cytokeratin 5/6.

7) The method according to claims 1 to 3, comprising detecting underexpression of at least one of the proteins:
Estrogen Receptor, FHIT, GATA3, Mucin 1, P-Cadherin, Progesterone receptor, TACC1, TACC2, TACC3, Afadin, Aurora A, a-Catenin, b-Catenin, BCL2, Cytokeratin 8/18 and E-Cadherin.

8) The method according to claim 4, comprising detecting underexpression of at least one of the proteins:
Estrogen Receptor, FHIT, GATA3, Mucin 1, P-Cadherin, Progesterone receptor, TACC 1, TACC2, TACC3, Afadin, Aurora A, a-Catenin, b-Catenin, BCL2, Cytokeratin 8/18 and E-Cadherin.

9) A protein library for molecular characterization of histopathologic features of breast disease comprising or corresponding to a pool of protein sequences, over or under expressed, in breast tissue or cells, the pool comprising at least one of a protein set comprising:
Afadin, Aurora A, a-Catenin, b-Catenin, BCL2, Cyclin D1, Cyclin E, Cytokeratin 5/6, Cytokeratin 8/18, E-Cadherin, EGFR, ERBB2, ERBB3, ERBB4, Estrogen receptor, FGFR1, FHIT, GATA3, Ki67, Mucin 1, P53, P-Cadherin, Progesterone receptor, TACC1, TACC2, TACC3, Cytokeratin 6, Cytokeratin 18, Ang1, AuroraB, BCRP1, CathepsinD, CD10, CD44, CK14, Cox2, FGF2, GATA4, Hif1a, MMP9, MTA1, NM23, NRG1a, NRG1beta, P27, Parkin, PLAU, S100, SCRIBBLE, Smooth Muscle Actin, THBS1 and TIMP1.

10) A protein library according to claim 7 immobilized on a solid support.

11) The protein library according to claim 8, wherein the support is selected from the group consisting of nylon membrane, nitrocellulose membrane, polyvinylidene difluoride, glass slide, glass beads, polyustyrene plates, membranes on glass support, silicon chip and gold chip.

12) A method for analyzing differential protein expression associated with histopathologic features of breast disease in breast tissues comprising:
a) obtaining breast tissue cells from a patient,
b) detecting overexpression or underexpression of a pool of proteins; and
c) measuring in the tissue cells obtained in step (a) over or underexpression of proteins of the library according to any of claims 9 to 11.

13) The method according to claim 12, wherein the proteins are directly or indirectly labeled before step (b).

14) The method according to claim 13, wherein the label is selected from the group consisting of radioactive, calorimetric, enzymatic, molecular amplification, bioluminescent and fluorescent labels.

15) The method according to claim 14, wherein one or more specific label(s) are used for each protein of the library.

16) The method according to claim 10, wherein measuring over or under expression of proteins is carried out on a tissue microarray.

17) The method according to claim 10, wherein measuring of over or under expression of protein is carried out by ImmunoHistoChemistry (IHC) technology.

18) A method according to claim 12, wherein detection of over or under expression of the pool of protein is alternatively carried out on breast tumor cell lines.

19) The method according to claim 10, further comprising:
a) obtaining a control sample
b) measuring in the control sample obtained in step (a) expression level of each protein corresponding to the library according to claim 9
c) comparing expression level of each protein with the level of equivalent protein in a tissue sample.

20) The method according to claim 1 for detecting, diagnosing, staging, monitoring, predicting, preventing conditions associated with breast cancer.

21) The method according to claim 1 for predicting clinical outcome of breast cancer.

22) The method according to claim 1 for predicting occurrence of metastatic relapse.

23) The method according to claim 20 for determining the stage or aggressiveness of a breast cancer.

24) A method according to claims 1 or 10, wherein a breast tissue sample is obtained from a patient regardless of whether the patient has received a neo adjuvant or an adjuvant therapy.

25) The method according to claim 24, wherein the breast tissue sample is obtained from a patient who has received an adjuvant therapy.

26) The method according to claim 24, wherein the breast tissue sample is obtained from a patient who has not received an adjuvant therapy.

27) A method for treating a patient with breast cancer comprising:
(i) analyzing differential protein expression associated with histopathologic features of breast cancer according to the method of claim 1 on a sample from the patient, and (ii) selecting a treatment for the patient based on analysis of differential protein expression profile obtained.

28) A method for treating a patient with breast cancer comprising:
(i) analyzing differential protein expression associated with histopathologic features of breast cancer according to the method of claim 10 on a sample from the patient, and (ii) selecting a treatment for the patient based on analysis of differential protein expression profile obtained.

29) The method according to claim 1, wherein detecting the overexpression or underexpression of the pool of protein in breast tissues comprises detecting overexpression or underexpression of nucleic acids coding for the proteins.

30) A nucleic acids library for molecular characterization of histopathologic features of breast disease comprising nucelic acids according to claim 29.
